(19) United States
(12) Patent Application Publication
(10) Pub. No.: US 2023/0000820 A1
(43) Pub. Date: Jan. 5, 2023

(54) SOLID DOSAGE FORM OF N-1-PYRROLIDINE-N-5-(3-TRIFLUOROMETHOXY)PHENYL BIGUANIDE AND USES THEREOF

(71) Applicant: ImmunoMet Therapeutics, Inc., Houston, TX (US)

(72) Inventors: Dean WELSCH, Houston, TX (US); Thomas E. RICHARDSON, Houston, TX (US)

(21) Appl. No.: 17/850,018

(22) Filed: Jun. 27, 2022

Related U.S. Application Data
(60) Provisional application No. 63/268,103, filed on Feb. 16, 2022.
(60) Provisional application No. 63/215,706, filed on Jun. 28, 2021.

(51) Int. Cl. A61K 31/40; A61K 31/4178; A61K 31/439; A61K 31/166; A61K 31/454; A61K 31/5415; A61K 31/495; A61K 9/28

(52) U.S. Cl. CPC A61K 31/40; A61K 31/4178; A61K 31/439; A61K 31/166; A61K 31/454; A61K 31/5415; A61K 31/495; A61K 9/28

(57) ABSTRACT

The invention features solid dosage forms of N-1-pyrrolidine-N-5-(3-trifluoromethoxy)phenyl biguanide, or a pharmaceutically acceptable salt thereof, where the solid dosage form includes an enteric coating and uses thereof.

### FIELD OF THE INVENTION

In general, the present invention relates to N-1-pyrroldine-N-5-(3-trifluoromethoxy)phenyl biguanide that is formulated in a solid dosage form for oral administration containing an enteric coating. The invention further relates to methods of treating a subject having a fibrosis-related disease. The invention further relates to methods of treating a subject having cancer.

### BACKGROUND OF THE INVENTION

Fibrosis, the accumulation of undesirable and harmful scar tissue, occurs in multiple tissues and organs and causes a variety of disorders, including pulmonary fibrosis, cystic fibrosis, idiopathic pulmonary fibrosis, fibrothorax, renal fibrosis, radiation-induced lung injury following treatment for cancer, bridging fibrosis, myocardial fibrosis such as interstitial fibrosis or replacement fibrosis, retroperitoneal fibrosis, nephrogenic systemic fibrosis, myelofibrosis, Peyronie's disease, keloid scar, arthrofibrosis, chronic kidney disease, adhesive capsulitis of the shoulder, arthrofibrosis, and systemic sclerosis. Idiopathic pulmonary fibrosis, the most common form of pulmonary fibrosis, affects approximately 130,000 individuals in the United States alone and approximately 5,000,000 individuals globally. Current therapies for treating fibrosis disorders are limited and cause severe side effects such as severe adverse gastrointestinal events. Accordingly, there is an ongoing need for improved therapies for treating fibrosis.

Cancer, a group of diseases caused by the harmful, abnormal, uncontrolled, and undesirable growth of cells, occurs in multiple tissues and organs, including the breasts, the lungs, the bladder, the colon, the rectum, the uterus, the testes, the kidneys, the blood, the lymphatic system, the liver, the bile ducts, the skin, the pancreas, the prostate, the thyroid gland, the brain, and the stomach. Globally, approximately 10,000,000 individuals die of cancer per year. Current therapies for treating cancer cause severe side effects such as severe adverse gastrointestinal events. Accordingly, there is an ongoing need for improved therapies for treating cancer.

### SUMMARY OF THE INVENTION

The present invention provides enteric coated solid dosage forms of N-1-pyrrolidine-N-5-(3-trifluoromethoxy)phenyl biguanide (“compound 1”), or a pharmaceutical salt thereof, and methods of treating fibrosis, particularly idiopathic pulmonary fibrosis, by administering an effective amount of compound 1 to a patient.

In a first embodiment, the invention features solid dosage forms of compound 1, or a pharmaceutically acceptable salt thereof. In some embodiments, including the foregoing embodiment, the invention features solid dosage forms of compound 1, or a pharmaceutically acceptable salt thereof, that include an enteric coating. In some embodiments, including any of the foregoing embodiments, the enteric coating is at least 1% of the weight of the composition (e.g., at least 1%, at least 2%, at least 3%, at least 4%, or at least 5%).

In some embodiments, including any of the foregoing embodiments, the invention features solid dosage forms of compound 1, or a pharmaceutically acceptable salt thereof, that are formulated for delayed release, e.g., a solid dosage form which contains an enteric coating. In some embodiments, including any of the foregoing embodiments, the solid dosage form of compound 1, or a pharmaceutically acceptable salt thereof, that includes an enteric coating is formulated for release distal to the stomach.

In some embodiments, including any of the foregoing embodiments, the invention features a pharmaceutical composition of compound 1 that includes an enteric coating that is formulated for oral administration.

In some embodiments, including any of the foregoing embodiments, the invention features a solid dosage form of compound 1, that includes an enteric coating, that additionally includes at least one excipient, for example a filler, and/or disintegrant, and/or binder, and/or glidant, and/or lubricant.

In some embodiments, including any of the forgoing embodiments, the invention features a pharmaceutical composition of the solid dosage form of compound 1 that includes an enteric coating.

In some embodiments, including any of the foregoing embodiments, the invention features the administration of the pharmaceutical composition of compound 1, or a pharmaceutically acceptable salt thereof, that includes an enteric coating, to a subject, wherein the administration results in a plasma concentration of compound 1 in the subject that is between 15 ng/mL and 1500 ng/mL 4-8 hours after administration (e.g., 4 hours, 5 hours, 6 hours, 7 hours, or 8 hours after administration). In some embodiments, administration results in a plasma concentration of compound 1 in a subject that is, e.g., between about 30 ng/mL and about 750 ng/mL, between about 50 ng/mL and about 250 ng/mL, between about 60 ng/mL and about 325 ng/mL, between about 120 ng/mL and about 180 ng/mL 4-8 hours after administration (e.g., 4 hours, 5 hours, 6 hours, 7 hours, 8 hours after administration).

In some embodiments, administration results in a plasma concentration of compound 1 in a subject that is between 15 ng/mL±1.5 ng/mL and 1500 ng/mL±150 ng/mL, e.g., between 30 ng/mL±3 ng/mL and 750 ng/mL±75 ng/mL, between about 50 ng/mL±5 ng/mL and about 250 ng/mL±25 ng/mL between 60 ng/mL±6 ng/mL and 325 ng/mL±32.5 ng/mL, between 120 ng/mL±12 ng/mL and 163±16.3 ng/mL 4-8 hours after administration (e.g., 4 hours, 5 hours, 6 hours, 7 hours, or 8 hours after administration). In some embodiments, administration results in a plasma concentration of compound 1 in a subject that is between 15 ng/mL and 1500 ng/mL, e.g., between 30 ng/mL and 750 ng/mL, between 50 ng/mL and 250 ng/mL, between 60 ng/mL and 325 ng/mL, between 120 ng/mL and 163 ng/mL, 4-8 hours after administration (e.g., 4 hours, 5 hours, 6 hours, 7 hours, or 8 hours after administration).

In some embodiments, including any of the foregoing embodiments, administration results in a plasma concentration of compound 1 in the subject that is greater than about 15 ng/mL 4-8 hours after administration (e.g., 4 hours, 5 hours, 6 hours, 7 hours, or 8 hours after administration). In some embodiments administration results in a plasma concentration of compound 1 in the subject that is greater than about 30 ng/mL 4-8 hours after administration (e.g., 4 hours, 5 hours, 6 hours, 7 hours, or 8 hours after administration) In some embodiments administration results in a plasma concentration of compound 1 in the subject that is greater than about 50 ng/mL 4-8 hours after administration (e.g., 4 hours, 5 hours, 6 hours, 7 hours, or 8 hours after administration). In some embodiments administration results in a plasma concentration of compound 1 in the subject that is greater than about 60 ng/mL 4-8 hours after administration (e.g., 4 hours, 5 hours, 6 hours, 7 hours, or 8 hours after administration). In some embodiments administration results in a plasma concentration of compound 1 in the subject that is greater than about 120 ng/mL 4-8 hours after administration (e.g., 4 hours, 5 hours, 6 hours, 7 hours, or 8 hours after administration). In some embodiments administration results in a plasma concentration of compound 1 in the subject that is greater than about 180 ng/mL 4-8 hours after administration (e.g., 4 hours, 5 hours, 6 hours, 7 hours, or 8 hours after administration). In some embodiments administration results in a plasma concentration of compound 1 in the subject that is greater than about 250 ng/mL 4-8 hours after administration (e.g., 4 hours, 5 hours, 6 hours, 7 hours, or 8 hours after administration). In some embodiments administration results in a plasma concentration of compound 1 in the subject that is greater than about 325 ng/mL 4-8 hours after administration (e.g., 4 hours, 5 hours, 6 hours, 7 hours, or 8 hours after administration). In some embodiments administration results in a plasma concentration of compound 1 in the subject that is greater than about 750 ng/mL 4-8 hours after administration (e.g., 4 hours, 5 hours, 6 hours, 7 hours, or 8 hours after administration). In some embodiments administration results in a plasma concentration of compound 1 in the subject that is greater than about 1500 ng/mL 4-8 hours after administration (e.g., 4 hours, 5 hours, 6 hours, 7 hours, or 8 hours after administration).

In some embodiments, including any of the foregoing embodiments, administration results in a plasma concentration of compound 1 in the subject that is greater than 15 ng/mL±1.5 ng/mL 4-8 hours after administration (e.g., 4 hours, 5 hours, 6 hours, 7 hours, or 8 hours after administration). In some embodiments administration results in a plasma concentration of compound 1 in the subject that is greater than 30 ng/mL±3.0 ng/mL 4-8 hours after administration (e.g., 4 hours, 5 hours, 6 hours, 7 hours, or 8 hours after administration) In some embodiments administration results in a plasma concentration of compound 1 in the subject that is greater than 50 ng/mL±5.0 ng/mL 4-8 hours after administration (e.g., 4 hours, 5 hours, 6 hours, 7 hours, or 8 hours after administration). In some embodiments administration results in a plasma concentration of compound 1 in the subject that is greater than 60 ng/mL±6.0 ng/mL 4-8 hours after administration (e.g., 4 hours, 5 hours, 6 hours, 7 hours, or 8 hours after administration). In some embodiments administration results in a plasma concentration of compound 1 in the subject that is greater than 120 ng/mL±12 ng/mL 4-8 hours after administration (e.g., 4 hours, 5 hours, 6 hours, 7 hours, or 8 hours after administration). In some embodiments administration results in a plasma concentration of compound 1 in the subject that is greater than 180 ng/mL±18 ng/mL 4-8 hours after administration (e.g., 4 hours, 5 hours, 6 hours, 7 hours, or 8 hours after administration). In some embodiments administration results in a plasma concentration of compound 1 in the subject that is greater than 250 ng/mL±25.0 ng/mL 4-8 hours after administration (e.g., 4 hours, 5 hours, 6 hours, 7 hours, or 8 hours after administration). In some embodiments administration results in a plasma concentration of compound 1 in the subject that is greater than 325 ng/mL±32.5 ng/mL 4-8 hours after administration (e.g., 4 hours, 5 hours, 6 hours, 7 hours, or 8 hours after administration). In some embodiments administration results in a plasma concentration of compound 1 in the subject that is greater than 750 ng/mL±75 ng/mL ng/mL 4-8 hours after administration (e.g., 4 hours, 5 hours, 6 hours, 7 hours, or 8 hours after administration). In some embodiments administration results in a plasma concentration of compound 1 in the subject that is greater than 1500 ng/mL±150 ng/mL 4-8 hours after administration (e.g., 4 hours, 5 hours, 6 hours, 7 hours, or 8 hours after administration).

In some embodiments, including any of the foregoing embodiments, administration results in a plasma concentration of compound 1 in the subject that is greater than 15 ng/mL 4-8 hours after administration (e.g., 4 hours, 5 hours, 6 hours, 7 hours, or 8 hours after administration). In some embodiments administration results in a plasma concentration of compound 1 in the subject that is greater than 30 ng/mL 4-8 hours after administration (e.g., 4 hours, 5 hours, 6 hours, 7 hours, or 8 hours after administration). In some embodiments administration results in a plasma concentration of compound 1 in the subject that is greater than 50 ng/mL 4-8 hours after administration (e.g., 4 hours, 5 hours, 6 hours, 7 hours, or 8 hours after administration). In some embodiments administration results in a plasma concentration of compound 1 in the subject that is greater than 60 ng/mL 4-8 hours after administration (e.g., 4 hours, 5 hours, 6 hours, 7 hours, or 8 hours after administration). In some embodiments administration results in a plasma concentration of compound 1 in the subject that is greater than 120 ng/mL 4-8 hours after administration (e.g., 4 hours, 5 hours, 6 hours, 7 hours, or 8 hours after administration). In some embodiments administration results in a plasma concentration of compound 1 in the subject that is greater than 180 ng/mL 4-8 hours after administration (e.g., 4 hours, 5 hours, 6 hours, 7 hours, or 8 hours after administration). In some embodiments administration results in a plasma concentration of compound 1 in the subject that is greater than 250 ng/mL 4-8 hours after administration (e.g., 4 hours, 5 hours, 6 hours, 7 hours, or 8 hours after administration). In some embodiments administration results in a plasma concentration of compound 1 in the subject that is greater than 325 ng/mL 4-8 hours after administration (e.g., 4 hours, 5 hours, 6 hours, 7 hours, or 8 hours after administration). In some embodiments administration results in a plasma concentration of compound 1 in the subject that is greater than 750 ng/mL 4-8 hours after administration (e.g., 4 hours, 5 hours, 6 hours, 7 hours, or 8 hours after administration). In some embodiments administration results in a plasma concentration of compound 1 in the subject that is greater than 1500 ng/mL 4-8 hours after administration (e.g., 4 hours, 5 hours, 6 hours, 7 hours, or 8 hours after administration).

In some embodiments, including any of the foregoing embodiments, the invention features the administration of the pharmaceutical composition of compound 1, or a pharmaceutically acceptable salt thereof, that includes an enteric coating, as one part of a combination therapy that includes the administration of an anti-emetic agent. In some embodiments, the anti-emetic agent is Ondansetron, Granisetron, Metoclopramide, Domperidone, Promethazine, or Meclizine.

In another aspect, the invention also provides a method of treating fibrosis in a subject in need thereof, the method including administering an effective amount of the solid dosage form of compound 1, or a pharmaceutically acceptable salt thereof, or the pharmaceutical composition of compound 1, or a pharmaceutically acceptable salt thereof, that includes an enteric coating, to the subject. In a related aspect, the invention provides a method of treating a pulmonary fibrosis, in particular idiopathic pulmonary fibrosis; cystic fibrosis; fibrothorax; renal fibrosis; radiation-induced lung injury following treatment for cancer; bridging fibrosis; myocardial fibrosis, in particular interstitial fibrosis or replacement fibrosis; retroperitoneal fibrosis; nephrogenic systemic fibrosis; myelofibrosis; Peyronie's disease; keloid scar; arthrofibrosis; chronic kidney disease; adhesive capsulitis of the shoulder; arthrofibrosis; or systemic sclerosis in a subject in need thereof, the method including administering an effective amount of the solid dosage form of compound 1, or a pharmaceutically acceptable salt thereof, or the pharmaceutical composition thereof, that includes an enteric coating, to the subject.

In another aspect, the invention also provides the solid dosage form of compound 1, or a pharmaceutically acceptable salt thereof, or the pharmaceutical composition of compound 1, or a pharmaceutically acceptable salt thereof, that includes an enteric coating, for use in a method of treating fibrosis in a subject in need thereof, the method including administering an effective amount of the solid dosage form of compound 1, or a pharmaceutically acceptable salt thereof, or the pharmaceutical composition of compound 1, or a pharmaceutically acceptable salt thereof, to the subject. In a related aspect, the invention provides the solid dosage form of compound 1, or a pharmaceutically acceptable salt thereof, or the pharmaceutical composition thereof, that includes an enteric coating, for use in a method of treating a pulmonary fibrosis, in particular idiopathic pulmonary fibrosis; cystic fibrosis; fibrothorax; renal fibrosis; radiation-induced lung injury following treatment for cancer; bridging fibrosis; myocardial fibrosis, in particular interstitial fibrosis or replacement fibrosis; retroperitoneal fibrosis; nephrogenic systemic fibrosis; myelofibrosis; Peyronie's disease; keloid scar; arthrofibrosis; chronic kidney disease; adhesive capsulitis of the shoulder; arthrofibrosis; or systemic sclerosis in a subject in need thereof, the method including administering an effective amount of the solid dosage form of compound 1, or a pharmaceutically acceptable salt thereof, or the pharmaceutical composition thereof, to the subject.

In another aspect, the invention also provides a method of treating cancer in a subject in need thereof, the method including administering an effective amount of the solid dosage form of compound 1, or a pharmaceutically acceptable salt thereof, or the pharmaceutical composition of compound 1, or a pharmaceutically acceptable salt thereof, that includes an enteric coating, to the subject. In a related aspect, the invention provides a method of treating breast cancer, lung cancer, bladder cancer, colon cancer, rectal cancer, uterine cancer, testicular cancer, kidney cancer, leukemia, lymphoma, hepatic cancer, cholangiocarcinoma, melanoma, pancreatic cancer, prostate cancer, thyroid cancer, glioblastoma, or gastric cancer in a subject in need thereof, the method including administering an effective amount of the solid dosage form of compound 1, or a pharmaceutically acceptable salt thereof, or the pharmaceutical composition thereof, that includes an enteric coating, to the subject.

In another aspect, the invention also provides the solid dosage form of compound 1, or a pharmaceutically acceptable salt thereof, or the pharmaceutical composition of compound 1, or a pharmaceutically acceptable salt thereof, that includes an enteric coating, for use in a method of treating cancer in a subject in need thereof, the method including administering an effective amount of the solid dosage form of compound 1, or a pharmaceutically acceptable salt thereof, or the pharmaceutical composition of compound 1, or a pharmaceutically acceptable salt thereof, to the subject. In a related aspect, the invention provides the solid dosage form of compound 1, or a pharmaceutically acceptable salt thereof, or the pharmaceutical composition thereof, that includes an enteric coating, for use in a method of treating breast cancer, lung cancer, bladder cancer, colon cancer, rectal cancer, uterine cancer, testicular cancer, kidney cancer, leukemia, lymphoma, hepatic cancer, cholangiocarcinoma, melanoma, pancreatic cancer, prostate cancer, thyroid cancer, glioblastoma, or gastric cancer in a subject in need thereof, the method including administering an effective amount of the solid dosage form of compound 1, or a pharmaceutically acceptable salt thereof, or the pharmaceutical composition thereof, to the subject.

In some embodiments, including any of the foregoing embodiments, administration of the solid dosage form of compound 1, or a pharmaceutically acceptable salt thereof, or the pharmaceutical composition thereof, that includes an enteric coating, to a subject does not result in an adverse gastrointestinal event or results in the reduction of an adverse gastrointestinal event relative to an alternative dosage form. In some embodiments, including any of the foregoing embodiments, the adverse gastrointestinal event is emesis.

In some embodiments, including any of the foregoing embodiments, administration of compound 1, or a pharmaceutically acceptably salt thereof, in the present solid dosage or pharmaceutical composition thereof, that includes an enteric coating, results an improved pharmacokinetic profile (e.g., greater compound 1 area under the curve) than administration of compound 1, or a pharmaceutically acceptably salt thereof, in alternative dosage forms. In some embodiments, including any of the foregoing embodiments, administration of compound 1, or a pharmaceutically acceptably salt thereof, in the present solid dosage form, that includes an enteric coating, or pharmaceutical composition thereof to a subject results in fewer adverse gastrointestinal events in the subject than administration of compound 1, or a pharmaceutically acceptably salt thereof, in an alternative dosage form. In some embodiments, including any of the foregoing embodiments, administration of compound 1, or a pharmaceutically acceptably salt thereof, that includes an enteric coating, in the present solid dosage form or pharmaceutical composition thereof to a subject results in fewer instances of emesis in the subject than administration of compound 1, or a pharmaceutically acceptably salt thereof, in an alternative dosage form.

### Definitions

In this application, unless otherwise clear from context, (i) the term “a” may be understood to mean “at least one”; (ii) the terms “comprising” and “including” may be understood to encompass itemized components or steps whether presented by themselves or together with one or more additional components or steps; and (iii) the term “approximately” may be understood to permit standard variation as would be understood by those of ordinary skill in the art; and (iv) where ranges are provided, endpoints are included.

As used herein, the term “about” represents a value that is in the range of ±10% of the value that follows the term “about.” Reference to “about” a value or parameter herein includes (and describes) variations that are directed to that value or parameter per se. For example, a description referring to “about X” includes the description of “X”.

As used herein, the term “administration” refers to the administration of a composition (e.g., a compound or a preparation that includes compound 1) to a subject or system. Administration to an animal subject (e.g., to a human) may be by any appropriate route. For example, in some embodiments, administration may be bronchial (including by bronchial instillation), buccal, enteral, interdermal, intra-arterial, intradermal, intragastric, intramedullary, intramuscular, intranasal, intraperitoneal, intrathecal, intravenous, intraventricular, mucosal, nasal, oral, rectal, subcutaneous, sublingual, topical, tracheal (including by intratracheal instillation), transdermal, vaginal, and vitreal.

An “effective amount” of a compound (e.g., compound 1) may vary according to factors such as the disease state, age, sex, and weight of the individual, and the ability of the compound to elicit the desired response. A therapeutically effective amount encompasses an amount in which any toxic or detrimental effects of the compound are outweighed by the therapeutically beneficial effects. An effective amount also encompasses an amount sufficient to confer benefit, e.g., clinical benefit.

As used herein, the term “subject” or “participant” or “patient” refers to any organism to which a compound or composition in accordance with the invention may be administered, e.g., for experimental, diagnostic, prophylactic, and/or therapeutic purposes. Typical subjects include any animal (e.g., mammals such as dogs, mice, rats, rabbits, pigs, non-human primates, and humans). A subject may seek or be in need of treatment, require treatment, be receiving treatment, be receiving treatment in the future, or be a human or animal who is under care by a trained professional for a particular disease or condition.

As used herein, the terms “treat,” “treated,” or “treating” mean both therapeutic treatment and prophylactic or preventative measures wherein the object is to prevent or slow down (lessen) an undesired physiological condition, disorder, or disease, or obtain beneficial or desired clinical results. Beneficial or desired clinical results include, but are not limited to, alleviation of symptoms; diminishment of the extent of a condition, disorder, or disease; stabilized (i.e., not worsening) state of condition, disorder, or disease; delay in onset or slowing of condition, disorder, or disease progression; amelioration of the condition, disorder, or disease state or remission (whether partial or total), whether detectable or undetectable; an amelioration of at least one measurable physical parameter, not necessarily discernible by the subject; or enhancement or improvement of condition, disorder, or disease. Treatment includes eliciting a clinically significant response without excessive levels of side effects. Treatment also includes prolonging survival as compared to expected survival if not receiving treatment.

In the digestive tract, the coated tablets pass through the stomach first. The pH of this region is approximately 1 to 3. The enteric coating component allows the medicament core to remain substantially intact and thus prevents the pharmacologically active substance from being released in this region or the acid from penetrating through the tablet core. The tablets then pass through the small intestine wherein the majority of the enteric coating component will dissolve and release the pharmacologically active substance therein. In normal flow direction, the small intestine consists of the duodenum, jejunum, and ilieum. The pH of these regions is approximately 5 to approximately 7.2.

As used herein “enteric coating” is a polymer material or materials which encase the medicament core (e.g., one containing N1-pyrrolidine-N5-(3-trifluoromethoxy)phenyl biguanide, (compound 1) or a pharmaceutically acceptable salt thereof) as they pass through the stomach. A suitable pH-sensitive polymer is one which will dissolve in the intestinal juices at the higher pH levels (pH greater than 4.5), such as within the small intestine and therefor permit release of the pharmacologically active substance in the regions of the small intestine and not in the upper portion of the GI tract, such as the stomach. Enteric coatings also protect the stomach from irritating pharmaceutical ingredients. Enteric coatings can be applied to a variety of formulations, including tablets, capsules, and microparticles. The enteric coating may be applied directly to the medicament core and may be an integral part of the tablet, capsule, or microparticle. Alternatively, the enteric coating may be separate from the medicament core where the coating, while encapsulating the medicament core, is not attached to the medicament core.

As used herein, “delayed release” means a pharmaceutical preparation, e.g., an orally administered formulation, which passes through the acidic environment of the stomach substantially intact and dissolves in the more basic environment of the small intestine. In some embodiments, delayed release of the active agent (e.g., N1-pyrrolidine-N5-(3-trifluoromethoxy)phenyl biguanide (compound 1) or a pharmaceutically acceptable salt thereof) results from the use of a pH-sensitive enteric coating of an oral dosage form. An enteric coating can be combined with, for example, a delayed release formulation so as to extend the period of time over which drug is released.

As used herein, “effective amount” means the amount of an agent sufficient to effect beneficial or desired results in a patient, such as disease remission, and, as such, an “effective amount” depends upon the context in which it is being applied, including the age and weight of the patient, the nature of the disease, including the disease-affected organ(s), the disease status or level of activity, and other factors.

As used herein, “pharmaceutically acceptable salt,” represents those salts which are, within the scope of sound medical judgment, suitable for use in contact with the tissues of humans and animals without undue toxicity, irritation, allergic response and the like, and are commensurate with a reasonable benefit/risk ratio. Pharmaceutically acceptable salts are well known in the art. For example, pharmaceutically acceptable salts are described in: Berge et al., J. Pharmaceutical Sciences 66:1-19, 1977 and in Pharmaceutical Salts: Properties, Selection, and Use, (Eds. P. H. Stahl and C. G. Wermuth), Wiley-VCH, 2008. The salts can be prepared in situ during the final isolation and purification of the compounds of the invention or separately by reacting the free base group with a suitable organic or inorganic acid. Representative acid addition salts include acetate, adipate, alginate, ascorbate, aspartate, benzenesulfonate, benzoate, bisulfate, bitartrate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts include sodium, lithium, potassium, calcium, magnesium, and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like.

As used herein, “adverse gastrointestinal event” means any undesired condition occurring in the gastrointestinal tract. Non-limiting examples include, nausea, emesis, constipation, diarrhea, flatulence, loss of appetite, mucositis, indigestion, abdominal pain, bloating, and ulcers. Adverse reactions are generally exacerbated at higher dose levels.

As used herein, “severe” means an event which is fatal, life threatening, results in hospitalization or prolongation of existing hospitalization, causes a persistent inability to participate in normal life activities, results in the inability to secure or continue employment, or requires intervention to prevent permanent impairment or damage.

Unless otherwise defined, all technical and scientific terms used herein have the same meaning as commonly understood by one of ordinary skill in the art to which this invention belongs. Methods and materials are described herein for use in the present disclosure; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

The details of one or more embodiments of the invention are set forth in the Drawings and Description below. Other features, objects, and advantages of the invention will be apparent from the Description and from the claims.

### BRIEF DESCRIPTION OF THE DRAWINGS

FIG. 1 is a graph showing the plasma concentrations of compound 1 in a dog model over time after administration of various formulations of compound 1.

FIG. 2 is a graph showing the plasma concentrations of compound 1 in human subjects over time after administrations of various doses of compound 1.

FIG. 3 is a graph showing the plasma concentrations of compound 1 in human subjects over time after administration of various doses of compound 1.

FIG. 4 is a chart showing the doses of compound 1 administered to human subjects during a phase 1 clinical trial of the safety and tolerability of compound 1.

FIG. 5 is a chart showing treatment-related adverse events reported by human subjects during a phase 1 clinical trial of the safety and tolerability of compound 1.

FIG. 6 is a chart showing treatment-related adverse events reported by human subjects during a phase 1 clinical trial of the safety and tolerability of compound 1.

FIG. 7 is a chart showing treatment-related adverse events reported by subjects during a phase 1 clinical trial of the safety and tolerability of compound 1.

### DETAILED DESCRIPTION OF THE INVENTION

Biguanide compounds may be used to treat fibrosis or cancer. However, the administration of previously-known dosage forms of biguanide compounds can have disadvantages including side effects such as adverse gastrointestinal events. For these reasons, there is a need for a biguanide-based substance effective at treating fibrosis or cancer that is formulated in a manner that minimizes side effects such as adverse gastrointestinal events.

The present inventors have found that administering biguanide substance N-1-pyrrolidine-N-5-(3-trifluoromethoxy)phenyl biguanide formulated in a solid dosage form for oral administration containing an enteric coating results in fewer adverse gastrointestinal events relative to administering N-1-pyrrolidine-N-5-(3-trifluoromethoxy)phenyl biguanide in standard tablet form. Therefore, the object of this invention is to provide a formulation of N-1-pyrrolidine-N-5-(3-trifluoromethoxy)phenyl biguanide that causes fewer undesirable side effects than other formulations of N-1-pyrrolidine-N-5-(3-trifluoromethoxy)phenyl biguanide.

Compound 1, N-1-pyrrolidine-N-5-(3-trifluoromethoxy)phenyl biguanide, is a functionalized biguanide that is useful in treating fibrosis or cancer. The present inventors have discovered that formulating compound 1, or a pharmaceutically acceptable salt thereof, in a solid dosage form for oral administration containing an enteric coating results in surprisingly high plasma concentration of compound 1 4-8 hours after administration to a subject, as well as surprisingly fewer adverse gastrointestinal events upon administration to a subject relative to the administration of compound 1, or a pharmaceutically acceptable salt thereof, in standard tablet form. Accordingly, the present invention features solid dosage forms compound 1, or a pharmaceutically acceptable salt thereof, containing an enteric coating, pharmaceutical compositions thereof, and methods for the treatment of fibrosis in a subject by administering the formulation of compound 1, or a pharmaceutically acceptable salt thereof.

### Solid Dosage Forms for Oral Administration

The pharmaceutical compositions contemplated by the invention include those formulated for oral administration (“oral dosage forms”). Oral dosage forms can be, for example, in the form of tablets, capsules, a liquid solution or suspension, a powder, or liquid or solid crystals, which contain the active ingredient(s) in a mixture with non-toxic pharmaceutically acceptable excipients. These compositions can be prepared in a variety of ways well known in the pharmaceutical art, and can be made so as to release compound 1, or a pharmaceutically acceptable salt thereof, in specific segments of the gastrointestinal tract at controlled times by a variety of excipients and formulation technologies. For example, formulations may be tailored to address a specific disease, to achieve plasma levels of compound 1 required to achieve therapeutic efficacy, to enable a desired duration of drug effect, and to provide a set of compositions with varying drug release.

The pharmaceutical compositions contemplated by the invention may include compound 1, or a pharmaceutically acceptable salt thereof, in a mixture with non-toxic pharmaceutically acceptable excipients. Pharmaceutically acceptable excipients are known to the skilled artisan. Excipients may be, for example, inert diluents or fillers such as sucrose, sorbitol, sugar, mannitol, microcrystalline cellulose, in particular microcrystalline cellulose PH101 or microcrystalline cellulose PH200, starches including potato starch, calcium carbonate, sodium chloride, lactose, calcium phosphate, calcium sulfate, or sodium phosphate; disintegrants such as crospovidone, sodium alginate, colloidal magnesiumaluminum silicate, calcium silicate, sodium starch glycolate, acrylic acid derivatives, microcrystalline cellulose, sodium carboxymethyl cellulose, calcium carboxymethyl cellulose, modified cellulose gum, cross-linked povidone, alginic acid and alginates, pregelatinised starch, modified corn starch cellulose derivatives including microcrystalline cellulose, starches including potato starch, croscarmellose sodium, alginates, or alginic acid; binders such as sucrose, glucose, sorbitol, acacia, alginic acid, gelatin, starch, pregelatinized starch, microcrystalline cellulose, magnesium aluminum silicate, carboxymethylcellulose sodium, methylcellulose, hydroxypropyl methylcellulose, hydroxypropyl cellulose EXF, ethylcellulose, polyvinylpyrrolidone, or polyethylene glycol; lubricants and/or glidants such as colloidal silicon dioxide, particularly colloidal silicon dioxide Cab-O-Sil® MSP, glycerol tribehenate, magnesium stearate, calcium stearate, talc, sodium stearyl fumarate, sodium behenate, stearic acid, cetyl alcohol, polyoxyethylene glycol, leucine, sodium benzoate, stearates, polyethylene glycol, glyceryl monostearate, glyceryl palmitostearate, liquid paraffin, poloxamer, sodium lauryl sulphate, magnesium lauryl sulphate, hydrogenated castor colloidal silicon dioxide, palmitostearate, stearic acid, zinc stearate, stearyl alcohol, silicas, or hydrogenated vegetable oil; anti-caking agents such as colloidal silicon dioxide, microcrystalline cellulose, tricalcium phosphate, microcrystalline cellulose, magnesium stearate, sodium bicarbonate, sodium ferrocyanide, potassium ferrocyanide, calcium ferrocyanide, calcium phosphate, sodium silicate, colloidal silicon dioxide, calcium silicate, magnesium trisilicate, talcum powder, sodium aluminosilicate, potassium aluminum silicate, calcium aluminosilicate, bentonite, aluminum silicate, stearic acid, polydimethylsiloxane. Other pharmaceutically acceptable excipients may be colorants, flavoring agents, plasticizers, humectants, and buffering agents.

Suitable pharmaceutical carriers, as well as pharmaceutical necessities for use in pharmaceutical formulations, are described in Remington: The Science and Practice of Pharmacy, 21st Ed., Gennaro, Ed., Lippencott Williams & Wilkins (2005), a well-known reference text in this field, and in the USP/NF (United States Pharmacopeia and the National Formulary) or corresponding European or Japanese reference documents.

### Enteric Coatings

The pharmaceutical compositions formulated for oral delivery of the present invention can be coated to provide a dosage form affording the advantage of delayed or extended release. The coating may be adapted to release the active drug substance in a predetermined pattern (e.g., in order to achieve a controlled release formulation) or it may be adapted not to release the active drug substance until after passage through the stomach, e.g., distal to the stomach, by use of an enteric coating (e.g., polymers that are pH-sensitive (“pH controlled release”), polymers with a slow or pH-dependent rate of swelling, dissolution or erosion (“time-controlled release”), polymers that are degraded by enzymes (“enzyme-controlled release” or “biodegradable release”) and polymers that form firm layers that are destroyed by an increase in pressure (“pressure-controlled release”)). Exemplary enteric coatings that can be used in the pharmaceutical compositions described herein include sugar coatings, film coatings (e.g., based on hydroxypropylmethylcellulose, methylcellulose, methyl hydroxyethylcellulose, hydroxypropylcellulose, carboxymethylcellulose, acrylate copolymers, polyethylene glycols and/or polyvinylpyrrolidone), or coatings based on methacrylic acid copolymer, cellulose acetate phthalate, hydroxypropyl methylcellulose phthalate, hydroxypropyl methylcellulose acetate succinate, polyvinyl acetate phthalate, shellac, and/or ethylcellulose. Furthermore, a time delay material such as, for example, glyceryl monostearate or glyceryl distearate, may be employed.

For delayed release formulations, (e.g., those containing an enteric coating) the therapeutic agent of the composition may be targeted for release distal to the stomach (e.g., in the small intestine). The formulation may contain an enteric coating such that the composition is resistant to the low pH environment found in the stomach, but sensitive to the higher pH environment of the small intestine. For example, some pH sensitive pharmaceutical coatings dissolve around pH 5.5, pH 6 or pH 6.5 in the duodenum, while other coatings dissolve around pH 7, which is more typical of the ileum; some types of colon-targeted formulations are composed, in part, of polymers which are refractory to digestion by human (or mammalian) enzymes but can be degraded by enzymes produced by enteric bacteria, thereby effecting release of therapeutic agents co-formulated with said polymers.

Alternatively, leaky enteric coatings may be used to provide a release profile intermediate between immediate release and delayed release formulations, e.g., those with an enteric coating. For example, U.S. patent application 20080020041 A1 discloses pharmaceutical formulations coated with an enteric material that releases at least a portion of an active ingredient upon contacting gastric fluid, with the remainder released upon contacting intestinal fluid.

Examples of commercial enteric coating technologies include but are not limited to: AcrylEZE, Opadry, Nutrateric and Sureteric products (Colorcon, West Point Pa.), Advantia Performance Specialty Coatings (International Specialty Products, Wayne N.J.), Kollicoat product line (BASF Corporation, Ludwigshafen Germany), Aquacoat products (FMC BioPolymer), Eastman C-A-P (Eastman Chemical Co. Kingsman Tenn.), Eudragit product line (Evonik Industries), and AQOAT, HP-50 and HP-55 product lines (Shin Etsu Pharma). Ashland Specialty Ingredients, Encap Drug Delivery, and Sanyo Chemical Industries, Ltd. also sell enteric coating systems. Examples of pH sensitive film forming polymers commonly used in enteric coated formulations include: (i) cellulose-based polymers such as cellulose acetate pthalate (e.g. Aquacoat CPD, FMC; C-A-P, Eastman Chemical Co.), cellulose acetate succinate, cellulose acetate trimellitate, hydroxypropylmethylcellulose pthalate, hydroxypropylmethylcellu lose acetate succinate (e.g. AquaSolve, Ashland Specialty Ingredients, Wilmington Del.); (ii) polymethacrylates such as poly(methacrylic acid-ethyl acrylate) (e.g. Eudragit L30D-55 and Eudragit L100-55, Evonik Industries; AcrylEZE, Colorcon; Kollicoat MAE 30 DP and Kollicoat MAE 100 P, BASF Pharma Ingredients and Services; Polyquid PA-30, Sanyo Chemical Industries) and poly(methacrylic acid-methyl methacrylate) in 1:1 and 1:2 ratios; (iii) polyvinyl derivatives such as poly(vinyl acetate) pthalate (e.g. Sureteric, Colorcon); and (iv) other copolymers such as half esters of the copolymer of styrene and maleic acid, half esters of the copolymer of vinyl ether and maleic acid, and copolymers of vinyl acetate and crotonic acid. Enteric coatings are also made using shellac (e.g., PROTECT, Sensient Pharmaceutical Coating Systems) or sodium alginate and zein (Encap Drug Delivery). Hydroxypropylmethylcellulose is also referred to as hypromellose or HPMC.

Multiple techniques for applying enteric coatings to pharmaceutical compositions are known in the art. In one non-limiting example, tablets and/or capsules may undergo a drum coating process in which they are tumbled in a cylindrical drum while being sprayed by a coating solution. In an additional non-limiting example, tablets and/or capsules may undergo a fluid-bed coating process in which air is passed over tablets or capsules at a sufficient velocity to separate the tables and/or capsules into individual units, at which point the tablets/capsules are sprayed with coating solution from above (e.g., top spray coating), from below (e.g., bottom spray coating), or from the side (e.g., HP spray coating). The amount of coating is determined by the amount of solution sprayed. The coating may be applied in a single application or may be built up in layers through the use of multiple applications.

The solid dosage forms and/or pharmaceutical compositions described herein may be provided with instructions for delivery including explanation of any color coding or alphanumeric text on the surface or packaging of the compositions, as well as instructions regarding whether the compositions should be ingested at certain times of day, or with food (e.g. specific types or amounts of food), liquids, a meal (including details about the type of meal) or other medications, and whether the patient should remain upright or sitting for some period of time after drug administration.

### Release Profile

Controlled release compositions for oral use may be constructed to release the active drug by controlling the dissolution and/or the diffusion of the active drug substance. Any of a number of strategies can be pursued in order to obtain controlled release and the targeted plasma concentration versus time profile. In one example, controlled release is obtained by appropriate selection of various formulation parameters and ingredients, including, e.g., various types of controlled release compositions and coatings. Examples include single or multiple unit tablet or capsule compositions, oil solutions, suspensions, emulsions, microcapsules, microspheres, nanoparticles, patches, and liposomes. In certain embodiments, compositions include biodegradable, pH, and/or temperature-sensitive polymer coatings (e.g., enteric coatings). Dissolution or diffusion controlled release can be achieved by appropriate coating of a tablet, capsule, pellet, or granulate formulation of compounds, or by incorporating the compound into an appropriate matrix. A controlled release coating may include one or more of the coating substances mentioned above.

In some embodiments, administration results in a plasma concentration of compound 1 in a subject that is between about 15 ng/mL and about 1500 ng/mL, e.g., between about 30 ng/mL and about 750 ng/mL, between about 50 ng/mL and about 250 ng/mL, between about 60 ng/mL and about 325 ng/mL, between about 120 ng/mL and about 180 ng/mL 4-8 hours after administration (e.g., 4 hours, 5 hours, 6 hours, 7 hours, 8 hours after administration). In some embodiments, administration results in a plasma concentration of compound 1 in a subject that is between 15 ng/mL±1.5 ng/mL and 1500 ng/mL±150 ng/mL, e.g., between 30 ng/mL±3 ng/mL and 750 ng/mL±75 ng/mL, between about 50 ng/mL±5 ng/mL and about 250 ng/mL±25 ng/mL between 60 ng/mL±6 ng/mL and 325 ng/mL±32.5 ng/mL, between 120 ng/mL±12 ng/mL and 163±16.3 ng/mL 4-8 hours after administration (e.g., 4 hours, 5 hours, 6 hours, 7 hours, or 8 hours after administration). In some embodiments, administration results in a plasma concentration of compound 1 in a subject that is between 15 ng/mL and 1500 ng/mL, e.g., between 30 ng/mL and 750 ng/mL, between 50 ng/mL and 250 ng/mL, between 60 ng/mL and 325 ng/mL, between 120 ng/mL and 163 ng/mL, 4-8 hours after administration (e.g., 4 hours, 5 hours, 6 hours, 7 hours, or 8 hours after administration).

### Adverse Gastrointestinal Effects

The solid dosage form of compound 1, or a pharmaceutically acceptable salt thereof, containing an enteric coating, or pharmaceutical composition thereof, reduces the occurrence of adverse gastrointestinal events when administered to a subject relative to the administration of compound 1, or a pharmaceutically acceptable salt thereof, alone or in alternative formulations or compositions. The solid dosage form of compound 1, or a pharmaceutically acceptable salt thereof, containing an enteric coating, or the pharmaceutical composition thereof, elicits fewer gastrointestinal events when administered to a subject relative to the administration of compound 1, or a pharmaceutically acceptable salt thereof, alone or in standard tablet form. Nonlimiting examples of adverse gastrointestinal events include nausea, emesis, constipation, diarrhea, flatulence, loss of appetite, mucositis, indigestion, abdominal pain, bloating, and ulcers.

### Dosages

The dosage of the composition used in the methods described herein can vary depending on many factors, e.g., the age, health, and weight of the recipient; the nature and extent of the symptoms; the frequency of the treatment, and the type of concurrent treatment, if any; and the clearance rate of the compound in the animal to be treated. One of skill in the art can determine the appropriate dosage based on the above factors. The composition used in the methods described herein may be administered initially in a suitable dosage that may be adjusted as required, depending on the clinical response. In general, a suitable daily dose of a compound of the invention will be that amount of the compound that is the lowest dose effective to produce a therapeutic effect. Such an effective dose will generally depend upon the factors described above.

While the attending physician ultimately will decide the appropriate amount and dosage regimen, an effective amount of a composition of the invention may be determined by the weight of the subject. For example, an effective amount may be between about 8 mg once a day (QD) and about 6,400 mg QD, e.g., between about 8 mg QD and about 3,200 mg QD, between about 8 mg QD and about 3,200 mg QD, between about 8 mg QD and about 1,200 mg QD, between about 8 mg QD and about 800 mg QD, e.g., between about 16 mg QD and about 400 mg QD, between about 32 mg QD and about 200 mg QD, between about 64 mg QD and 100 mg QD, between about 100 mg QD and 400 mg QD, between about 200 mg QD and about 300 mg QD, between about 400 mg QD and about 1,200 mg QD, between about 600 mg QD and about 1,000 mg QD, between about 700 mg QD and about 900 mg QD, between about 750 mg QD and about 850 mg QD, of a solid dosage form or composition described herein. In some embodiments, an effective amount may be between 8 mg QD±0.8 mg QD and 6,400 mg QD±640 mg QD, 8 mg QD±0.8 mg QD and 3,200 mg QD±320 mg QD, 8 mg QD±0.8 mg QD and 2,400 mg QD±240 mg QD, 8 mg QD±0.8 mg QD and 1,200 mg QD±160 mg QD, 8 mg QD±0.8 mg QD and 800 mg QD±80 mg QD, between 16 mg QD±1.6 mg QD and 400 mg QD±40 mg QD, between 32 mg QD±3.2 mg QD and 200 mg QD±20 mg QD, between 64 mg QD±6.4 mg QD and 100 mg QD±10 mg QD, between 100 mg QD±10 mg QD and 400 mg QD±40 mg QD, between 200 mg QD±20 mg QD and 300 mg QD±30 mg QD, between 400 mg QD±40 mg and 1,200 mg QD±120 mg, between 600 mg QD±60 mg and 1,000 mg QD±100 mg, between 700 mg QD±70 mg and 900 mg QD±90 mg, between 750 mg QD±75 mg and 850 mg QD±80 mg of a solid dosage form or composition described herein. In some embodiments, an effective amount may be between 8 mg QD and 6,400 mg QD, between 8 mg QD and 3,200 mg QD, between 8 mg QD and 2,400 mg QD, 8 mg QD and 1,200 mg QD, between 8 mg QD and 800 mg QD, between 16 mg QD and 400 mg QD, between 32 mg QD and 200 mg QD, between 64 mg QD and 100 mg QD, between 100 mg QD and 400 mg QD, between 200 mg QD and 300 mg QD, between 400 mg QD and 1,200 mg QD, between 600 mg QD and 1,000 mg QD, between 700 mg QD and 900 mg QD, between 750 mg QD and 850 mg QD of a solid dosage form or composition described herein.

In some embodiments, it may be a dose of about 8 mg QD of a solid dosage form or composition described herein. In some embodiments, it may be a dose of about 16 mg QD of a solid dosage form or composition described herein. In some embodiments, it may be a dose of about 32 mg QD of a solid dosage form or composition described herein. In some embodiments, it may be a dose of about 64 mg QD of a solid dosage form or composition described herein. In some embodiments, it may be a dose of about 100 mg QD of a solid dosage form or composition described herein. In some embodiments, it may be a dose of about 200 mg QD of a solid dosage form or composition described herein. In some embodiments, it may be a dose of about 300 mg QD of a solid dosage form or composition described herein. In some embodiments, it may be a dose of about 400 mg QD of a solid dosage form or composition described herein. In some embodiments, it may be a dose of about 800 mg QD of a solid dosage form or composition described herein. In some embodiments, it may be a dose of about 1,200 mg QD of a solid dosage form or composition described herein. In some embodiments, it may be a dose of about 2,400 mg QD of a solid dosage form or composition described herein. In some embodiments, it may be a dose of about 3,200 mg QD of a solid dosage form or composition described herein. In some embodiments, it may be a dose of about 6,400 mg QD of a solid dosage form or composition described herein.

In some embodiments, it may be a dosage of 8 mg QD±0.8 mg QD of a solid dosage form or composition described herein. In some embodiments, it may be a dosage of 16 mg QD±1.6 mg QD of a solid dosage form or composition described herein. In some embodiments, it may be a dosage of 32 mg QD±3.2 mg QD of a solid dosage form or composition described herein. In some embodiments, it may be a dosage of 64 mg QD±6.4 mg QD of a solid dosage form or composition described herein. In some embodiments, it may be a dosage of 100 mg QD±10 mg QD of a solid dosage form or composition described herein. In some embodiments, it may be a dosage of 200 mg QD±20 mg QD of a solid dosage form or composition described herein. In some embodiments, it may be a dosage of 300 mg QD±30 mg QD of a solid dosage form or composition described herein. In some embodiments, it may be a dosage of 400 mg QD±40 mg QD of a solid dosage form or composition described herein. In some embodiments, it may be a dosage of 800 mg QD±80 mg QD of a solid dosage form or composition described herein. In some embodiments, it may be a daily dosage of 1,200 mg QD±120 mg of a solid dosage form or composition described herein. In some embodiments, it may be a daily dosage of 2,400 mg QD±240 mg of a solid dosage form or composition described herein. In some embodiments, it may be a daily dosage of 3,200 mg±320 mg of a solid dosage form or composition described herein. In some embodiments, it may be a daily dosage of 6,400 mg±640 mg of a solid dosage form or composition described herein.

In some embodiments, it may be a dosage of 8 mg QD of a solid dosage form or composition described herein. In some embodiments, it may be a dosage of 16 mg QD of a solid dosage form or composition described herein. In some embodiments, it may be a dosage of 32 mg QD of a solid dosage form or composition described herein. In some embodiments, it may be a dosage of 64 mg QD of a solid dosage form or composition described herein. In some embodiments, it may be a dosage of 100 mg QD of a solid dosage form or composition described herein. In some embodiments, it may be a dosage of 200 mg QD of a solid dosage form or composition described herein. In some embodiments, it may be a dosage of 300 mg QD of a solid dosage form or composition described herein. In some embodiments, it may be a dosage of 400 mg QD of a solid dosage form or composition described herein. In some embodiments, it may be a dosage of 800 mg QD of a solid dosage form or composition described herein. In some embodiments, it may be a dosage of 1,200 mg QD of a solid dosage form or composition described herein. In some embodiments, it may be a dosage of 2400 mg QD of a solid dosage form or composition described herein. In some embodiments, it may be a dosage of 3200 mg QD of a solid dosage form or composition described herein. In some embodiments, it may be a dosage of 6400 mg QD of a solid dosage form or composition described herein.

In some embodiments, it may be a daily dosage of about 1 mg/kg to about 80 mg/kg of a solid dosage form or composition described herein. In some embodiments, it may be a daily dosage of about 1 mg/kg to about 40 mg/kg of a solid dosage form or composition described herein. In some embodiments, it may be a daily dosage of about 3 mg/kg to 30 mg/kg of a solid dosage form or composition described herein. In some embodiments, it may be a daily dosage of about 5 mg/kg to about 25 mg/kg of a solid dosage form or composition described herein. In some embodiments, it may be a daily dosage of about 10 mg/kg to about 20 mg/kg of a solid dosage form or composition described herein. In some embodiments, it may be a daily dosage of about 12 mg/kg to about 18 mg/kg of a solid dosage form or composition described herein. In some embodiments, it may be a daily dosage of about 1.25 mg/kg to about 15 mg/kg of a solid dosage form or composition described herein. In some embodiments, it may be a daily dosage of about 2.5 mg/kg to about 10 mg/kg of a solid dosage form or composition described herein. In some embodiments, it may be a daily dosage of about 5 mg/kg to about 8 mg/kg of a solid dosage form or composition described herein. In some embodiments, it may be a daily dosage of 1 mg/kg±0.1 mg/kg to 80 mg/kg±8300.0 mg/kg of a solid dosage form or composition described herein. In some embodiments, it may be a daily dosage of 1 mg/kg±0.1 mg/kg to 40 mg/kg±4.0 mg/kg of a solid dosage form or composition described herein. In some embodiments, it may be a daily dosage of 3 mg/kg±0.3 mg/kg of a solid dosage form or composition described herein to 30 mg/kg±3 mg/kg of a solid dosage form or composition described herein. In some embodiments, it may be a daily dosage of 5 mg/kg±0.5 mg/kg to about 25±2.5 mg/kg of a solid dosage form or composition described herein. In some embodiments, it may be a daily dosage of 10 mg/kg±1.0 mg/kg to about 20±2 mg/kg of a solid dosage form or composition described herein. In some embodiments, it may be a daily dosage of 12 mg/kg±1.2 mg/kg to 18 mg/kg±1.8 mg/kg of a solid dosage form or composition described herein. In some embodiments, it may be a daily dosage of 1.25±0.125 mg/kg to 15±1.5 mg/kg of a solid dosage form or composition described herein. In some embodiments, it may be a daily dosage of 2.5 mg/kg±0.25 to 10±1.0 mg/kg of a solid dosage form or composition described herein. In some embodiments, it may be a daily dosage of 5±0.5 mg/kg to 8±0.8 mg/kg of a solid dosage form or composition described herein. In some embodiments, it may be a daily dosage of 1 mg/kg to 40 mg/kg of a solid dosage form or composition described herein. In some embodiments, it may be a daily dosage of 3 mg/kg to 30 mg/kg of a solid dosage form or composition described herein. In some embodiments, it may be a daily dosage of 5 mg/kg to 25 mg/kg of a solid dosage form or composition described herein. In some embodiments, it may be a daily dosage of 10 mg/kg to 20 mg/kg of a solid dosage form or composition described herein. In some embodiments, it may be a daily dosage of 12 mg/kg to 18 mg/kg of a solid dosage form or composition described herein. In some embodiments, it may be a daily dosage of 1.25 mg/kg to 15 mg/kg of a solid dosage form or composition described herein. In some embodiments, it may be a daily dosage of 2.5 mg/kg to 10 mg/kg of a solid dosage form or composition described herein. In some embodiments, it may be a daily dosage of 5 mg/kg to 8 mg/kg of a solid dosage form or composition described herein. In some embodiments, it may be a daily dosage of about 30 mg/kg. In some embodiments, it may be a daily dose of about 50 mg/kg. In some embodiments, it may be a daily dose of about 60 mg/kg.

### Compositions Including Anti-Emetic Agents

In some embodiments, compound 1, or a pharmaceutically acceptable salt thereof, may be administered with an anti-emetic agent. Non-limiting examples of anti-emetic agents that may be administered with compound 1 include Ondansetron, Granisetron, Metoclopramide, Domperidone, Promethazine, and Meclizine.

In some embodiments, the anti-emetic agent is co-administered to a subject with the compound 1. In some embodiments, the anti-emetic agent and compound 1 are administered sequentially. In some embodiments, the anti-emetic agent is administered before compound 1. In some embodiments, compound 1 is administered before the anti-emetic agent. In some embodiments, the anti-emetic agent may be formulated in the same pharmaceutical composition as compound 1, or a pharmaceutically acceptable salt thereof. In alternative embodiments, the second anti-emetic agent may be in a separate pharmaceutical composition from 1, or a pharmaceutically acceptable salt thereof. For example, the composition can include an inner dosage and an outer dosage component, the latter being in the form of an envelope over the former. The two components can be separated by an enteric layer which serves to resist disintegration in the stomach and permit the inner component, e.g., containing compound 1, or a pharmaceutically acceptable salt thereof, to pass intact into the small intestine or to be delayed in release.

### Methods of Treatment

Compositions of compound 1, or a pharmaceutically acceptable salt thereof, may be used to treat fibrosis. Fibrosis is a form of pathological wound healing characterized by the overgrowth, hardening, and/or scarring of diverse tissues. This overgrowth, hardening, and/or scarring is attributed to the excess deposition of extracellular matrix components including collagen. Fibrosis is the end result of chronic inflammatory reactions induced by a variety of stimuli including persistent infections, autoimmune reactions, allergic responses, chemical injury, radiation, and tissue injury.

Pulmonary fibrosis is fibrosis that occurs in pulmonary tissue, e.g., the respiratory system. Idiopathic pulmonary fibrosis is pulmonary fibrosis, that is of an unknown cause. It is characterized by thickening and stiffening of lung tissue. Patients commonly experience shortness of breath and a dry cough. Complications of idiopathic pulmonary fibrosis may include pulmonary hypertension, heart failure, pneumonia, or pulmonary embolism.

Thus, the compositions of compound 1, or a pharmaceutically acceptable salt thereof, can be administered to a subject in an effective amount to treat fibrosis. Non-limiting examples of fibrosis that may be treated by a solid dosage form of compound 1, or a pharmaceutically acceptable salt thereof, include pulmonary fibrosis, cystic fibrosis, idiopathic pulmonary fibrosis, fibrothorax, renal fibrosis, radiation-induced lung injury following treatment for cancer, bridging fibrosis, myocardial fibrosis such as interstitial fibrosis or replacement fibrosis, retroperitoneal fibrosis, nephrogenic systemic fibrosis, myelofibrosis, Peyronie's disease, keloid scar, arthrofibrosis, chronic kidney disease, adhesive capsulitis of the shoulder, arthrofibrosis, or systemic sclerosis.

Compositions of compound 1, or a pharmaceutically acceptable salt thereof, may be used to treat cancer. Cancer is a group of diseases characterized by the harmful, abnormal, uncontrolled, and undesirable growth of cells. In some embodiments, the uncontrolled growth is due to cells that divide and proliferate in the absence of signals (e.g., growth factors) instructing them to do so. In some embodiments, the uncontrolled growth is due to cells which fail to respond to signals instructing them to stop growing and/or engage in programmed cell death (i.e., apoptosis). In some embodiments, cancer cells may spread throughout the body (i.e., metastasize). In some embodiments, cancer cells may form tumors. In some embodiments, cancer cells may form solid tumors.

The following description of compound 1 activity is provided without wishing to be bound by theory. In some embodiments, cancer cells have metabolic activity, e.g., Krebs cycle and oxidative phosphorylation (OXPHOS) activity, that is unique from the metabolic activity, e.g., Krebs cycle and OXPHOS activity, of non-cancer cells. In some embodiments, compound 1 targets the unique metabolic activity of cancer cells. In some embodiments, targeting the unique metabolic activities of cancer cells allows compound 1 to selectively kill cancer cells over non-cancer cells.

Thus, the compositions of compound 1, or a pharmaceutically acceptable salt thereof, can be administered to a subject in an effective amount to treat cancer. Non-limiting examples of cancer that may be treated by a solid dosage form of compound 1, or a pharmaceutically acceptable salt thereof, include breast cancer, lung cancer, bladder cancer, colon cancer, rectal cancer, uterine cancer, testicular cancer, kidney cancer, leukemia, lymphoma, hepatic cancer, cholangiocarcinoma, melanoma, pancreatic cancer, prostate cancer, thyroid cancer, glioblastoma, or gastric cancer.

### Equivalents and Scope

Those skilled in the art will recognize or be able to ascertain using no more than routine experimentation, many equivalents to the specific embodiments in accordance with the invention described herein. The scope of the present invention is not intended to be limited to the above Description.

The following Examples are illustrative only and not intended to limit the invention in any way.

### EXAMPLES

### Example 1: A Pharmacokinetic Study of N-1-pyrrolidine-N-5-(3-trifluoromethoxy)phenyl biguanide via IV and PO Administration in Beagle Dogs

### Dosing Information

Animals were dosed with N-1-pyrrolidine-N-5-(3-trifluoromethoxy)phenyl biguanide acetate salt as specified in Table 1. Dosages were freshly prepared on the day of dosing. The PO (per os) solution was DI water. The enteric capsule was HMPC enteric-coated capsules. The test substance was prepared and used immediately before administration. The preparation method was to weigh N-1-pyrrolidine-N-5-(3-trifluoromethoxy)phenyl biguanide and add sterile distilled water. The mixture was titrated to final volumes by mixing via sonication for 2 minutes and vortexing for 5 minutes. During the administration time, the preparation was handled at room temperature. All animals for IV administration had free access to food and water. Animals for PO were fasted overnight prior to dosing and were fed approximately 2 hours after dosing. Group 2 shared the same 2 animals. Animals in PO_A arm were dosed first. After 2 days washout period, animals in PO_B arm were dosed. After 2 days washout period, animals in PO_C arm were dosed. Clinical observations of animals treated with compound 1 were recorded as specified in Tables 3-5.

TABLE 1
Dosing Information for pharmacokinetic study of N-1-pyrrolidine-N-5-(3-trifluoromethoxy)phenyl
biguanide via PO administration in Beagle dogs.

| | | Dose Level | Dose Volume | Concentration | Administration | No. of |
|---|---|---|---|---|---|---|
| Group | Treatment | (mg/kg) | (mL/kg | (mg/mL) | Route | Animals |
| 1 | compound 1 | 10 | 2 | 5 | IV | 2 male |
| 2 | compound 1 | 100 Enteric | #capsules/dog | — | PO_A | 2 male |
| | | coated casing | | | | |
| | compound 1 | 100 | 10 | 10 | PO_B | |
| | Ondansetran | Ondansetran | 1 mg/Kg | — | PO_C | |
| | (Zofran) | (Zofran) | Tablet | | | |
| | T = −1 hr then | 1.0 | | | | |
| | compound 1 | | | | | |

### Pharmacokinetic Schedule

Plasma for both IV and PO dosing was sampled from animals pre-dose, and 5 minutes, 15 minutes, 30 minutes, 1 hour, 2 hours, 4 hours, 8 hours, and 24 hours post dose.

### Blood Sample Collection and Processing

Samples were collected via venipuncture of peripheral veins except the dosing vein. 0.3 mL of blood was drawn per time point. The anticoagulant used was potassium EDTA. Whole blood samples were gently inverted several times to ensure anticoagulation then placed on wet ice prior to centrifugation. Within 30 minutes of collection, the samples were centrifuged at approximately 2000×g (force) for 10 minutes at 2 to 8° C. to obtain plasma. The resultant plasma samples were immediately separated divided into 2 aliquots (˜75 μμ each) and transferred to cryogenic vial. Samples were maintained on wet ice throughout processing and stored in a freezer at −75±15° C. prior to analysis.

### Bioanalytical Criteria

The standard curve was run in duplicate with a minimum of six standards, and a minimum of five standards and the LLOQ should fall within ±20% of the nominal value. Verapamil and dexamethasone were normally used as internal standards. The lower limit of quantitation (LLOQ) had a minimum signal to noise ratio of 3. A minimum of duplicate QC's at three concentrations (low, mid, and high QC) were incorporated into each run with the low QC no more than 3×LLOQ, the mid QC around the middle of the curve, and the high QC should be near the ULOQ (minimally 80% but less than 100% of the value of the highest standard) for the run and the mean value should be within ±20% of the theoretical value. The results of the QC's provide the basis for accepting or rejecting the run. At least 67% or four of six QC's should be within 20% of their respective nominal values; 33% of the QC's (not replicates of the same concentration) can fall outside 20% of nominal value. The simplest model that adequately describes the concentration-response relationship should be used. Linear or quadratic regression can be used. Weighting was 1/x or 1/x2.

### PK Samples Analysis

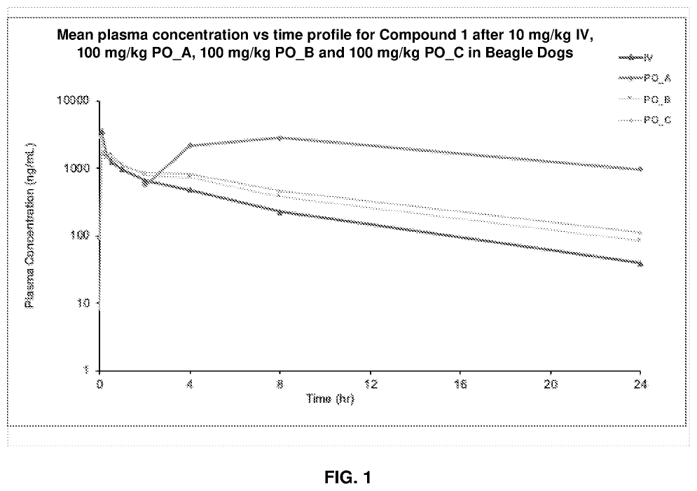

Concentrations of Test Article in the plasma samples were analyzed using a LC-MS/MS method. WinNonlin (Phoenix™, version 6.1) or other similar software will be used for pharmacokinetic calculations. The following pharmacokinetic parameters were be calculated, whenever possible from the plasma concentration versus time data: PO administration: T_{1/2}, Cₘₐₓ, Tₘₐₓ, MRT_{inf}, AUC_{inf}, AUCₗₐₛₜ, F%, Number of Points for Regression (FIG. 1). The pharmacokinetic data will be described using descriptive statistics such as mean, standard deviation. Additional pharmacokinetic or statistical analysis was performed at the discretion of the contributing scientist. The results are summarized in Tables 2, 3, 4, and 5.

TABLE 2
A table showing PK data of various formulations of compound 1 administered to beagle dogs.

| | IV | PO A | PO B | PO C |
|---|---|---|---|---|
| Dose | 10 mg/kg | 100 mg/kg | 100 mg/kg | 100 mg/kg |
| Formulation | DI Water | HPMC Enteric | DI Water | DI water + |
| | | Coating Capsule | | 1 mg Zofran |
| PK parameters | | Mean | Mean | Mean |

| | | | | |
|---|---|---|---|---|
| T_{1/2} | h | NA | 6.65 | 7.10 |
| Tₘₐₓ | h | 5.00 | 0.0830 | 0.292 |
| Cₘₐₓ | ng/mL | 2949 | 2820 | 1820 |
| AUCₗₐₛₜ | h*ng/mL | 43518 | 9946 | 11272 |
| AUC_{Inf} | h*ng/mL | NA | 10750 | 12452 |
| AUCₗₐₛₜ/D | h*mg/mL | 435 | 99 | 113 |
| F | | 61 | 14.4 | 16.7 |
| Notes | | Improved AUC | Initial | Zofran is an anti-emetic |
| | | Improved | Formulation | given prior to compound 1 |
| | | Bioavailability | and Dosing | dosing |
| | | Improved | | no difference in PK |
| | | Tolerability | | no difference in |
| | | | | tolerability |
| | | | | supports that compound |
| | | | | 1is NOT CNS triggered |
| | | | | emesis |

TABLE 3
A table showing clinical observations of dogs treated with compound 1 via intravenous
administration or via administration of a composition including an enteric coating.
Clinical Observation/Cageside Observation

| | | IV-1 | IV-2 | POA-3 | POA-4 |
|---|---|---|---|---|---|
| Observation | Explanation | 8312543 | 8311032 | 8310744 | 8319440 |
| 1. Tremor (slight) | Violent uncontrollable muscle contraction | No | No | No | No |
| 2. Uncoordinated movement | Abnormal movement | No | No | No | No |
| 3. Discoloration | Color different than the normal color | No | No | No | No |
| 4. Torpidity | Inactive compared to other | No | No | No | No |
| 5. Temperature Drop | Temperature below normal | No | No | No | No |
| 6. Temperature Rise | Temperature above normal | No | No | No | No |
| 7. Feces with abnormal content | N/A | No | No | No | No |
| 8. Dyspnea (labored breathing) | Difficult Breathing | No | No | No | No |
| 9. Frothy | Producing foam | No | No | No | No |
| 10. Soft Stool | Soft feces | No | No | No | No |
| 11. Loose feces | Soft with no solid pieces | No | No | Yes(f) | Yes(f) |
| 12. Emesis | Vomiting or vomited food residue found | No *(g) | Yes(a, e) | Yes(b, d) | Yes(c) |
| | in collection pan | | | | |
| 13. Diarrhea | Slight: No shape, soft, not like water | No | No | No | Yes(c) |
| | Moderate: No shape, like water | | | | |
| 14. Few feces | The absence of feces within 24 hrs | No | No | No | No |
| 15. Reduced food intake | Moderate: more than ⅔ food was left | No | No | No | No |
| 16. Weight drop | Obviously drop | No | No | No | No |
| 17. Comment present | N/A | No | No | No | No |

Key: “Yes” Observed; “No” Not Observed
a. Loose feces was observed from IV-2(8311032) at 5 min timepoint
b. Some broken capsule was found in the loose feces at 30 min timepoint from POA-3
c. “Loose feces” and “emesis” were observed from POA-4(8319440) at 4 h timepoint
d. “Loose feces” was observed from POA-3(8310744) at 8 h timepoint
e. Loose feces was observed from IV-2(8311032) at >4 h timepoint
f. Soft stool was observed from POA-3(8310744) and POA-4(8319440) at 24 h timepoint
g. Wrote over

TABLE 4
A table showing clinical observations of dogs treated with compound 1
via administration of a composition not including an enteric coating.
Clinical Observation/Cageside Observation

| | | POB-3 | POB-4 |
|---|---|---|---|
| Observation | Explanation | 8310744 | 8319440 |
| 1. Tremor (slight) | Violent uncontrollable muscle contraction | No | No |
| 2. Uncoordinated movement | Abnormal movement | No | No |
| 3. Discoloration | Color different than the normal color | No | No |
| 4. Torpidity | Inactive compared to other | No | No |
| 5. Temperature Drop | Temperature below normal | No | No |
| 6. Temperature Rise | Temperature above normal | No | No |
| 7. Feces with abnormal content | N/A | No | No |
| 8. Dyspnea (labored breathing) | Difficult Breathing | No | No |
| 9. Frothy | Producing foam | No | No |
| 10. Soft Stool | Soft feces | No | No |
| 11. Loose feces | Soft with no solid pieces | No | No |
| 12. Emesis | Vomiting or vomited food residue found in | Yes | Yes |
| | collection pan | | |
| 13. Diarrhea | Slight: No shape, soft, not like water | No | No |
| | Moderate: No shape, like water | | |
| 14. Few feces | The absence of feces within 24 hrs | No | No |
| 15. Reduced food intake | Moderate: more than ⅔ food was left | No | No |
| 16. Weight drop | Obviously drop | No | No |
| 17. Comment present | N/A | No | No |

Key: “Yes” Observed; “No” Not Observed
a. Emesis was observed from POB-3(8310744) at 5 min timepoint
b. Emesis was observed from POB-4(819440) at 2 min timepoint
c. Emesis was observed from POB-3(8310744) at 12 min timepoint

TABLE 5
A table showing clinical observations of dogs treated with an anti-emetic agent and
compound 1 via administration of a composition not including an enteric coating.
Clinical Observation/Cageside Observation

| | | POC-3 | POC-4 |
|---|---|---|---|
| Observation | Explanation | 8310744 | 8319440 |
| 1. Tremor (slight) | Violent uncontrollable muscle contraction | No | No |
| 2. Uncoordinated movement | Abnormal movement | No | No |
| 3. Discoloration | Color different than the normal color | No | No |
| 4. Torpidity | Inactive compared to other | No | No |
| 5. Temperature Drop | Temperature below normal | No | No |
| 6. Temperature Rise | Temperature above normal | No | No |
| 7. Feces with abnormal content | N/A | No | No |
| 8. Dyspnea (labored breathing) | Difficult Breathing | No | No |
| 9. Frothy | Producing foam | No | No |
| 10. Soft Stool | Soft feces | No | No |
| 11. Loose feces | Soft with no solid pieces | Yes(c) | Yes(d) |
| 12. Emesis | Vomiting or vomited food residue found in | Yes(a) | Yes(b) |
| | collection pan | | |
| 13. Diarrhea | Slight: No shape, soft, not like water | No | No |
| | Moderate: No shape, like water | | |
| 14. Few feces | The absence of feces within 24 hrs | No | No |
| 15. Reduced food intake | Moderate: more than ⅔ food was left | No | No |
| 16. Weight drop | Obviously drop | No | No |
| 17. Comment present | N/A | No | No |

Key: “Yes” Observed; “No” Not Observed
a. Emesis was observed from POC-3(8310744) at 2 min timepoint after dosing
b. Emesis was observed from POC-4(819440) at 1 min timepoint after dosing
c. Watery feces was observed from POC-3(8310744) at 6 min, 9 min, 10 min, and 11 min after dosing
d. Loose feces was observed from POC-4(8319440) at 8 h timepoint

### Example 2: A Pharmacokinetic Study of N-1-pyrrolidine-N-5-(3-trifluoromethoxy)phenyl biguanide via PO Administration in Beagle Dogs

### Test System

Male beagle dogs (non-naïve) were obtained from Jiangsu Johnsen Bioresource CO. and/or Beijing Rixinkeji CO., LTD and/or Marshall. The dogs were approximately 8 months to 3 years old and weighed approximately 9.0 kg to 13.0 kg at the day of dosing.

### Dosing Information

Animals were dosed with N-1-pyrrolidine-N-5-(3-trifluoromethoxy)phenyl biguanide acetate salt as specified in Table 6. Dosages were freshly prepared on the day of dosing. The PO solution was DI water. The enteric capsule was HMPC enteric-coated capsules. The test substance was prepared and used immediately before administration. The preparation method was to weigh N-1-pyrrolidine-N-5-(3-trifluoromethoxy)phenyl biguanide and add sterile distilled water. The mixture was titrated to final volumes by mixing via sonication for 2 minutes and vortexing for 5 minutes. During the administration time, the preparation was handled at room temperature. Animals were fasted overnight prior to dosing and were fed approximately 2 hours after dosing.

TABLE 6
Dosing Information for pharmacokinetic study of N-1-pyrrolidine-N-5-(3-trifluoromethoxy)phenyl
biguanide via PO administration in Beagle dogs.

| | Dose | Dose | | | | | | | |
|---|---|---|---|---|---|---|---|---|---|
| | Level | Volume | Conc. | Dog 1 | Dog 2 | Dog 3 | Dog 4 | Dog 5 | Dog 6 |
| Day | (mg/kg) | (mL/kg) | (mg/mL) | (male) | (female) | (male) | (female) | (male) | (female) |
| 1 | 3 | 5 | 0.6 | ENTERIC | SOLUTION | — | — | — | — |
| | | | | CAPSULE | | | | | |
| 4 | | | | SOLUTION | ENTERIC | — | — | — | — |
| | | | | | CAPSULE | | | | |
| 1 | 10 | | 2 | — | — | ENTERIC | SOLUTION | — | — |
| | | | | | | CAPSULE | | | |
| 4 | | | | — | — | SOLUTION | ENTERIC | — | — |
| | | | | | | | CAPSULE | | |
| 1 | 30 | | 6 | — | — | — | — | ENTERIC | SOLUTION |
| | | | | | | | | CAPSULE | |
| 4 | | | | — | — | — | — | SOLUTION | ENTERIC |
| | | | | | | | | | CAPSULE |

### Pharmacokinetic Schedule

Plasma was sampled from animals pre-dose, and 5 minutes, 15 minutes, 30 minutes, 1 hour, 2 hours, 4 hours, 8 hours, and 24 hours post dose.

### Blood Sample Collection and Processing

Samples were collected via venipuncture of peripheral veins except the dosing vein. 0.3 mL of blood was drawn per time point. The anticoagulant used was potassium EDTA. Whole blood samples were gently inverted several times to ensure anticoagulation then placed on wet ice prior to centrifugation. Within 30 minutes of collection, the samples were centrifuged at approximately 2000×g (force) for 10 minutes at 2 to 8° C. to obtain plasma. The resultant plasma samples were immediately separated divided into 2 aliquots (−75 μL each) and transferred to cryogenic vial. Samples were maintained on wet ice throughout processing and stored in a freezer at −75±15° C. prior to analysis.

### Bioanalytical Criteria

The standard curve was run in duplicate with a minimum of six standards, and a minimum of five standards and the LLOQ should fall within ±20% of the nominal value. Verapamil and dexamethasone were normally used as internal standards. The lower limit of quantitation (LLOQ) had a minimum signal to noise ratio of 3. A minimum of duplicate QC's at three concentrations (low, mid, and high QC) were incorporated into each run with the low QC no more than 3×LLOQ, the mid QC around the middle of the curve, and the high QC near the ULOQ (minimally 80% but less than 100% of the value of the highest standard) for the run and the mean value within ±20% of the theoretical value. The results of the QC's provided the basis for accepting or rejecting the run. At least 67% or four of six QC's were within 20% of their respective nominal values; 33% of the QC's (not replicates of the same concentration) fell outside 20% of nominal value. The simplest model that adequately described the concentration-response relationship was used. Linear or quadratic regression was used. Weighting was 1/x or 1/x2.

### PK Samples Analysis

Concentrations of Test Article in the plasma samples were analyzed using a LC-MS/MS method. WinNonlin (Phoenix™, version 6.1) or other similar software was used for pharmacokinetic calculations. The following pharmacokinetic parameters were calculated, whenever possible from the plasma concentration versus time data: PO administration: T_{1/2}, Cₘₐₓ, Tₘₐₓ, MRT_{inf}, AUC_{inf}, AUCₗₐₛₜ, F%, Number of Points for Regression. The pharmacokinetic data was described using descriptive statistics such as mean, standard deviation. Additional pharmacokinetic or statistical analysis was performed at the discretion of the contributing scientist. Results are summarized in Tables 7 and 8.

TABLE 7
A table showing PK data of various dosages of compound 1 administered to male beagle dogs.
Male (J A## Dog ID)

| | Dose | | | | | |
|---|---|---|---|---|---|---|
| | 3 mg/kg | | 10 mg/kg | | 30 mg/kg | |
| | Formulation | | | | | |
| | Enteric | | Enteric | | Enteric | |
| | capsule | DI H20 | capsule | DI H20 | capsule | DI H20 |
| | PK parameters | | | | | |
| | JA0039 | JA0039 | JA0025 | JA0025 | JA0031 | JA0031 |
| T_{1/2} | 5.29 | 5.68 | 5.92 | 6.79 | 7.64 | 7.21 |
| Tₘₐₓ | 1.00 | 1.00 | 2.00 | 0.500 | 2.00 | 0.0830 |
| Cₘₐₓ | 202 | 164 | 273 | 417 | 331 | 259 |
| AUCₗₐₛₜ | 985 | 816 | 2088 | 2176 | 3806 | 1459 |
| AUC_{Inf} | 1023 | 856 | 2223 | 2350 | 4253 | 1599 |
| AUC_%Extrap_obs | 3.68 | 4.70 | 6.04 | 7.38 | 10.5 | 8.78 |
| MRT_{Inf}_obs | 6.29 | 6.37 | 8.23 | 7.90 | 10.1 | 8.69 |
| AUCₗₐₛₜ/D | 328 | 272 | 209 | 218 | 127 | 48.6 |

| | | | |
|---|---|---|---|
| enteric/non-enteric | 1.2× | 1 | 2.6×× |

TABLE 8
A table showing PK data of various dosages of compound 1 administered
to female beagle dogs.
Female

| | 3 mg/kg | | 10 mg/kg | | 30 mg/kg | |
|---|---|---|---|---|---|---|
| | Enteric | | Enteric | | Enteric | |
| | capsule | DI H20 | capsule | DI H20 | capsule | DI H20 |
| | PK parameters | | | | | |
| | JA0100 | JA0100 | JA0022 | JA0022 | JA0056 | JA0056 |
| T_{1/2} | 6.34 | 7.25 | 7.19 | 6.00 | NA | 5.95 |
| Tₘₐₓ | 1.00 | 0.250 | 2.00 | 2.00 | 4.00 | 0.0830 |
| Cₘₐₓ | 220 | 85.1 | 328 | 107 | 950 | 619 |
| AUCₗₐₛₜ | 1185 | 393 | 2045 | 819 | 13170 | 3305 |
| AUC_{Inf} | 1271 | 431 | 2267 | 867 | NA | 3499 |
| AUC_%Extrap_obs | 6.80 | 8.90 | 9.80 | 5.50 | NA | 5.52 |
| MRT_{Inf}_obs | 7.77 | 8.75 | 9.65 | 7.32 | NA | 7.25 |
| AUCₗₐₛₜ/D | 395 | 131 | 204 | 81.9 | 439 | 110 |

| | | | |
|---|---|---|---|
| enteric/non-enteric | 3× | 2.5× | 4× |

### Example 3: Non-GLP Cross Over Pharmacokinetic Study Following Compound 1 Administration as an Oral Tablet or Capsule Containing the Tablet in Male and Female Beagle Dogs

### Objective

To evaluate the pharmacokinetics of compound 1 following cross over administration of an oral (PO) tablet or enteric capsule (containing the tablet) twice per phase to male and female beagle dogs.

### Capsule Information

Fillable clear empty vegan acid resistance capsules (Size 00), stored at room temperature and sourced from PureCaps USA.

### Test System

Beagle dogs sourced from Marshall BioResources (North Rose, N.Y.), between 2 and 3.5 years old, and weighing 6-12 kg.

### Selection for Study and Randomization

Animals that were judged acceptable for the study based on satisfactory pre-study acclimation and general health were assigned to the study. A general physical examination, including a body weight measurement, was conducted on all animals prior to assignment to the study. Animals were ranked by body weight and assigned to treatment groups using a computerized randomization assignment.

### Acclimation

Animals were washed out for a period of at least ten days prior to initiation of Phase 1 dose administration and at least two days between the end of phase 1 and the beginning of Phase 2.

### Justification for Route of Administration and Dosages

The purpose of this study was to characterize compound 1 exposure and to compare vomitus findings following compound 1 administration either as a tablet (within an acid-resistant capsule) or the tablet as is. Oral is the intended route of administration in humans. The dosages were selected based on experience with test article, including in prior 28-day GLP-compliant toxicity studies.

### Study Design

Compound 1 administration occurred twice per phase (once daily for two days) by PO tablet or was administered to male and female beagle dogs as described in Table 9. Administration occurred twice per phase (once daily for two days) by PO tablet or capsule (containing the tablet).

On days 1 and 2 (Phase 1) one group was administered the tablets as is and the other group was administered a tablet within a capsule (size 00 containing the tablet). Each group received the other form of test article (cross over design) on days 5 and 6 (Phase 2).

TABLE 9
A table showing group designation and compound 1 dosage levels.

| | | | | | | | Number | # |
|---|---|---|---|---|---|---|---|---|
| | | | | | Dose | Dose | of | Animals |
| | | Animal | Test | Route of | Level | concentration | Tablets | per |
| Phase | Group | IDs | Article | Administration | (mg/dog) | (mg/tablet) | per dog | group |
| 1 | 1 | 1M001- | Compound | PO Tablet | 400 | 400 | 1 | 2 males; |
| | | 1M002 | 1 | | | | | 1 female |
| | | 1F001 | | | | | | |
| | 2 | 2M001- | Compound | PO Capsule | 400 | 400 | 1 | 2 males; |
| | | 2M002 | 1 | (Containing the | | | | 1 female |
| | | 2F001 | | tablet) | | | | |
| 2 | 1 | 1M001 - | Compound | PO Capsule | 400 | 400 | 1 | 2 males; |
| | | 1M002 | 1 | (containing the | | | | 1 female |
| | | 1F001 | | tablet) | | | | |
| | 2 | 2M001- | Compound | PO Tablet | 400 | 400 | 1 | 2 males; |
| | | 2M002 | 1 | | | | | 1 female |
| | | 2F001 | | | | | | |

### Antemortem Evaluations

Animals were observed and data was recorded as indicated in Table 10.

TABLE 10
A table describing the evaluations of animals

| Procedure | Frequency |
|---|---|
| Cageside Observations | ≥twice daily on dosing days |
| (general signs of toxicity | (Days 1, 2, 5, 6) at approximately |
| including fecal and urine | 1 hour postdose and again at least |
| quality) | ix hours after the first observation. |
| | ≥once daily on Days 3, 4, and 7. |
| Physical Examinations | At the time of randomization. |
| (examined for general | |
| appearance, reaction to | |
| touch, muscle tone, | |
| respiration, fur, eyes, ears, | |
| nose, mouth, genitals, | |
| and anus of animal for | |
| abnormalities; examined | |
| for general body integument | |
| and palpated for | |
| masses, paying particular | |
| attention to the axillary, | |
| inguinal, abdominal, and | |
| ventrocervical areas) | |
| Body Weights | At the time of randomization; |
| | Just prior to dosing on the days of dose |
| | administration (Days 1, 2, 5, and 6). |

### Pharmacokinetics

The systemic exposure to compound 1 was evaluated in accordance with the collection schedule and procedures described in Table 11 and Sample analysis described in Table 12. Pharmacokinetic analysis was conducted using Phoenix WinNonlin software (version 8.1 or higher) using non-compartmental approach based on the route of administration. The following parameters were calculated for groups 1 and 2 whenever possible and as data allowed: Cₘₐₓ, Tₘₐₓ, and AUC. Additional parameters were calculated as appropriate. Descriptive statistics were generated using Phoenix WinNonlin. Key pharmacokinetic and phenotypic results are summarized in Tables 13 to 15.

TABLE 11
A table describing blood sample collection,
processing, storage, and shipment.

| | |
|---|---|
| Collection Intervals: | Two intervals (Following dose |
| | administration on the first day |
| | of each phase; Day 1 and Day 5) |
| Collection Time Points: | Predose (Day 5 only), 0.5, 1, 2, 4, |
| | 8, 12 and 24 hours postdose |
| Animals/Time Point: | Six |
| Collection Volume: | Approximately 0.5-1 mL of |
| | whole blood |
| Collection Site and Method: | Cephalic vein (Jugular as an |
| | alternative) |
| Anticoagulant: | K₂EDTA |
| Sample Requirements: | Samples were collected in K₂EDTA |
| | tubes and centrifuged under |
| | refrigerated conditions to collect |
| | plasma. The plasma was frozen |
| | at −70° C. until transferred for |
| | bioanalysis. An attempt was made |
| | to collect a terminal PK sample from |
| | animals euthanized moribund. |

TABLE 12
A table describing pharmacokinetic sample analysis

| | |
|---|---|
| Matrix: | Canine plasma |
| Bioanalytical Method: | LC-MS/MS |
| Analytes Assayed: | Compound 1, internal standard (Proguanil) |
| Samples Analyzed: | Both groups (all samples) |

TABLE 13
A table summarizing phenotypic data of beagle dogs
treated with alternative dosage forms of compound 1.

| | | | Compound 1 |
|---|---|---|---|
| Compound 1 | Emesis | Diarrhea | in plasma |
| presentation | (episodes) | (incidence) | (ug*hr/mL) |
| Clinical Tablet | 14 | 100% | 5.3 |
| Clinical Table in Enteric | 4 | 17% | 8.3 |
| Coated Capsule | | | |
| Net Effect | 71% | 83% | 55% |
| | reduction | reduction | increase |

TABLE 14
A table showing phenotypic data of dogs treated with alternative dosage forms of compound 1.

| | Day 1 | | | | | | | Day 5 | | | | | | |
|---|---|---|---|---|---|---|---|---|---|---|---|---|---|---|
| | | | | AUC | Day 2 | | | | | | AUC | Day 6 | | |
| | Form- | | | (ug* | Form- | | Diar- | Form- | | Diar- | (ug* | Form- | | Diar- |
| Dog | ulation | Emesis | Diarrhea | hr/mL) | ulation | Emesis | rhea | ulation | Emesis | rhea | hr/mL) | ulation | Emesis | rhea |
| 1M001 | Tablet | 4× in 1st | 1× in 1st | 4.206 | Tablet | 2×, 20-40 | 1× | Tablet in | None | None | 23.671 | Tablet in | None | None |
| | | 60′, 30- | 60′ | | | mL total | | Capsule | | | | Capsule | | |
| | | 60 mL | | | | | | | | | | | | |
| | | total | | | | | | | | | | | | |
| 1M002 | Tablet | 2× in 1st | 1× in 1st | 2.794 | Tablet | 1×, | 1× | Tablet in | None | 2× | 3.24 | Tablet in | 1×, 0-5 | None |
| | | 30′, 20- | 30′ | | | 10-20 mL | | Capsule | | | | Capsule | mL | |
| | | 40 mL | | | | | | | | | | | total | |
| | | total | | | | | | | | | | | | |
| 1F001 | Tablet | 2× in 1st | 1× @ 1st | 0.448 | Tablet | 1×, 5-10 | None | Tablet in | 1×, 0-5 | None | 5.932 | Tablet in | 1×, 0-5 | 1× |
| | | 30′, 10- | observation | | | mL total | | Capsule | mL | | | Capsule | mL | |
| | | 20 mL | | | | | | | total | | | | total | |
| | | total | | | | | | | | | | | | |
| 2M001 | Tablet in | 1× @ | None | 5.389 | Tablet in | None | None | Tablet | 1×, 0-5 | 1× | 14.947 | Tablet | 1×, 5- | None |
| | Capsule | 110′, 0-5 | | | Capsule | | | | mL | | | | 10 mL | |
| | | mL total | | | | | | | total | | | | total | |
| 2M002 | Tablet in | 1× @ | None | 2.833 | Tablet in | 1×, 0-5 | 1× | Tablet | 4×, 20- | 2× | 4.931 | Tablet | 2×, 20- | None |
| | Capsule | 45′, 0-5 | | | Capsule | mL total | | | 40 mL | | | | 40 mL | |
| | | mL total | | | | | | | total | | | | total | |
| 2F001 | Tablet in | 1× @ | None | 8.753 | Tablet in | 1×, 5-10 | None | Tablet | 1×, 0-5 | 1× | 4.724 | Tablet | 1×, 10- | 1× |
| | Capsule | 110′, 10- | | | Capsule | mL total | | | mL | | | | 20 mL | |
| | | 20 mL | | | | | | | total | | | | total | |
| | | total | | | | | | | | | | | | |

TABLE 15
A table showing phenotypic and pharmacokinetic data of dogs treated with alternative dosage forms of compound 1.

| | | Emesis | | | | Diarrhea | | | Mean | SEM |
|---|---|---|---|---|---|---|---|---|---|---|
| | | | Mean | Total | Mean | | | Mean | AUC | AUC |
| Form- | | Total | Occurrence | Volume | Volume | | Total | Occurrence | (ug* | (ug* |
| ulation | Incidence | Occurrences | per Dog | (mL) | per Dog | Incidence | Occurrences | per Dog | hr/mL) | hr/mL) |
| Tablet | 100% | 14 | 2.3 | 80-170 mL | 13-28 mL | 100% | 7 | 1.2 | 5.342 | 2.037 |
| Tablet in | 67% | 4 | 0.7 | 10-35 mL | 2-6 mL | 17% | 2 | 0.3 | 8.303 | 3.194 |
| Capsule | | | | | | | | | | |

| | | | | | |
|---|---|---|---|---|---|
| 33% redn | 71% redn | 80-88% redn | 83% redn | 71% redn | 55% |
| | | | | | increase |

### Example 3. Phase 1 Safety and Tolerability Study of Compound 1 in Human Subjects

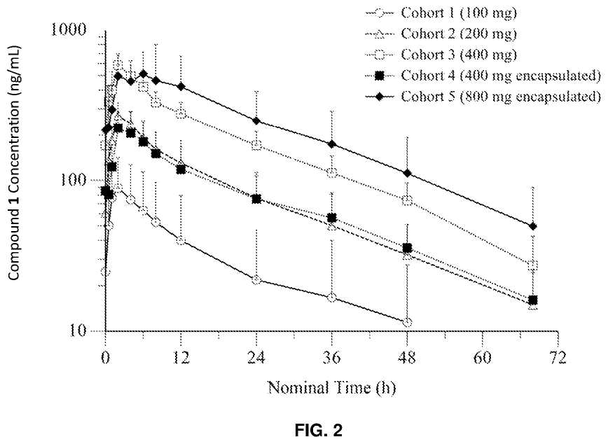

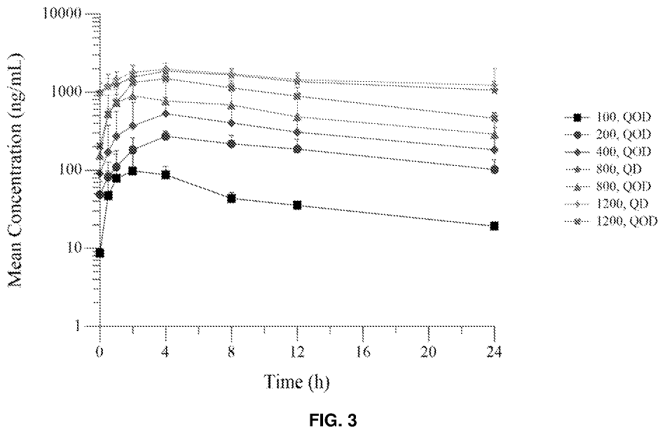

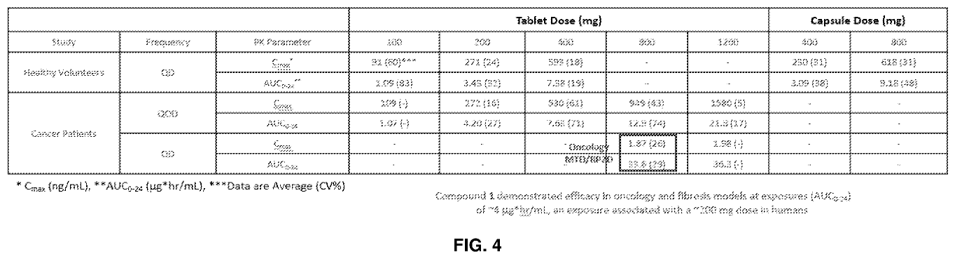

A phase 1 clinical study investigating the safety and tolerability of compound 1 in human subjects was conducted. Healthy volunteers were administered either an uncoated tablet at 100 mg QD (cohort 1, N=6), 200 mg QD (cohort 2, N=6), or 400 mg QD (cohort 3, N=6), or a coated tablet at 400 mg (cohort 4, N=6) or 800 mg QD (cohort 5, N=6), and the compound 1 plasma concentration achieved by the subjects was recorded over time (FIGS. 2 and 4). Cancer patients were administered either an uncoated tablet at 100 mg QOD (cohort 1, N=3), 200 mg QOD (cohort 2, N=3), 400 mg QOD (cohort 3, N=3), 800 mg QOD (cohort 4, N=3), or 1200 mg QOD (cohort 5, N=3), or were administered an uncoated tablet at 800 mg QD (cohort 6, N=3) or 1200 mg QD (cohort 7, N=4), and the compound 1 plasma concentration achieved by the subjects was recorded over time (FIGS. 3 and 4).

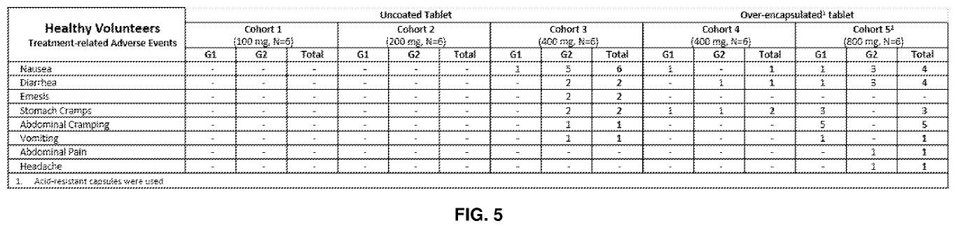

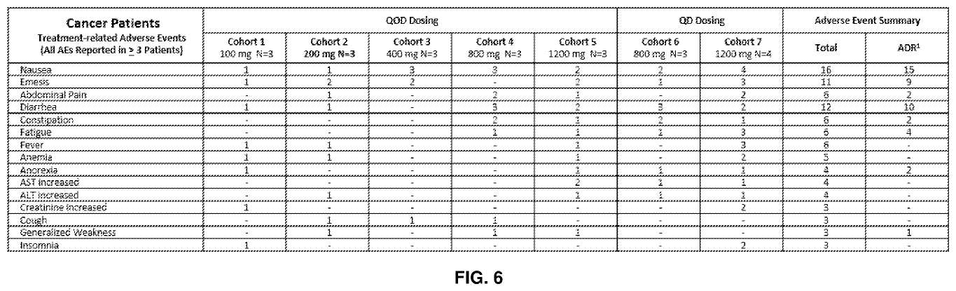

Compound 1 was well tolerated in healthy volunteers and cancer patients. No adverse events were recorded for cohort 1 or cohort 2 of healthy patients. Low-grade gastrointestinal adverse events were prominent in healthy volunteers and cancer patients (FIGS. 5 and 6).

### Example 4. Phase 1 Safety and Tolerability Study of Compound 1 in Human Subjects

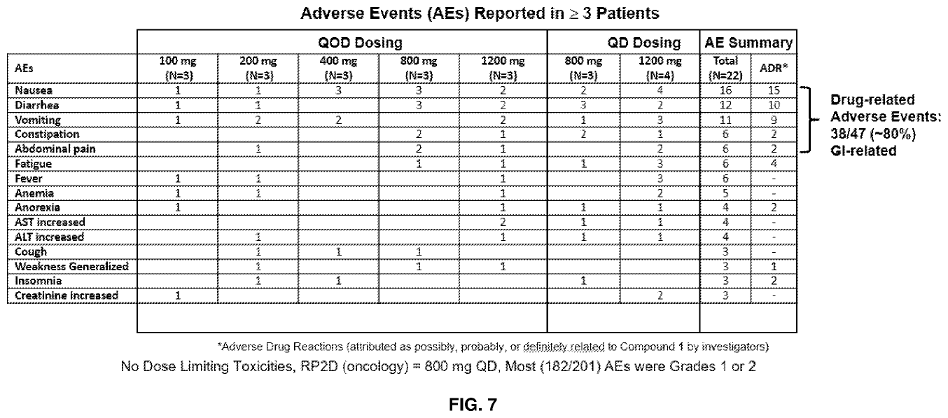

A phase 1 clinical study investigating the safety and tolerability of compound 1 in human subjects was conducted. 22 subjects with solid tumors were enrolled and split into seven cohorts. Cohort 1 received 100 mg QOD, cohort 2 received 200 mg QOD, cohort 3 received 400 mg QOD, cohort 4 received 800 mg QOD, cohort 5 received QOD, cohort 6 received 1,200 mg QOD, cohort 6 received 800 mg QD, and cohort 7 received 1,200 mg QD. Compound 1 was well tolerated in subjects, and the adverse events that were reported were primarily GI, low-grade, monitorable, manageable, and reversible (FIG. 7). This study found no dose limiting toxicities, and suggested that an 800 mg QD dose would be suitable for phase 2 studies.

### Numbered Embodiments

1. A solid dosage form of N-1-pyrrolidine-N-5-(3-trifluoromethoxy)phenyl biguanide, or a pharmaceutically acceptable salt thereof, wherein the solid dosage form is formulated for delayed release.
2. The solid dosage form of embodiment 1, wherein the solid dosage form comprises an enteric coating.
3. The solid dosage form of embodiment 1 or 2, wherein the N-1-pyrrolidine-N-5-(3-trifluoromethoxy)phenyl biguanide, or pharmaceutically acceptable salt thereof, is formulated for release distal to the stomach, to one or more regions of the intestine.
4. The solid dosage form of embodiment 3, in which the onset of N-1-pyrrolidine-N-5-(3-trifluoromethoxy)phenyl biguanide release occurs at or above pH 6 when distal to the stomach.
5. The solid dosage form of any one of embodiments 1 to 4, wherein the solid dosage form further comprises at least one pharmaceutically acceptable excipient such as a filler, disintegrant, binder, glidant, and/or lubricant.
6. A pharmaceutical composition comprising the solid dosage form of any one of embodiments 1 to 5.
7. The pharmaceutical composition of embodiment 6, wherein the pharmaceutical composition is formulated for administration with an anti-emetic agent.
8. The pharmaceutical composition of embodiments 6 or 7, wherein the anti-emetic agent is Ondansetron, Granisetron, Metoclopramide, Domperidone, Promethazine, or Meclizine.
9. The pharmaceutical composition of any one of embodiments 6 to 8, wherein administration of the composition to a subject results in a circulating plasma concentration of N-1-pyrrolidine-N-5-(3-trifluoromethoxy)phenyl biguanide that between 50 and 250 ng/mL for 4-8 hours following administration.
10. A method of treating fibrosis in a subject in need thereof, the method comprising administering an effective amount of the solid dosage form of any one of embodiments 1 to 5 or the pharmaceutical composition of any one of embodiment 6 to 9 to the subject.
11. The method of embodiment 10, wherein the pharmaceutical composition is administered orally.
12. The method of embodiment 10 or 11, wherein the fibrosis is a pulmonary fibrosis, in particular idiopathic pulmonary fibrosis; cystic fibrosis; fibrothorax; renal fibrosis; radiation-induced lung injury following treatment for cancer; bridging fibrosis; myocardial fibrosis, in particular interstitial fibrosis or replacement fibrosis; retroperitoneal fibrosis; nephrogenic systemic fibrosis; myelofibrosis; Peyronie's disease; keloid scar; arthrofibrosis; chronic kidney disease; adhesive capsulitis of the shoulder; arthrofibrosis; or systemic sclerosis.
13. A method of treating cancer in a subject in need thereof, the method comprising administering an effective amount of the solid dosage form of any one of claims 1 to 5 or the pharmaceutical composition of any one of claims 6 to 9 to the subject.
14. The method of embodiment 13, wherein the pharmaceutical composition is administered orally.
15. The method of embodiment 14, wherein the cancer is breast cancer, lung cancer, bladder cancer, colon cancer, rectal cancer, uterine cancer, testicular cancer, kidney cancer, leukemia, lymphoma, hepatic cancer, cholangiocarcinoma, melanoma, pancreatic cancer, prostate cancer, thyroid cancer, glioblastoma, or gastric cancer.
16. The method of any one of embodiments 10 to 15, wherein administration to a subject does not result in an adverse gastrointestinal event.
17. The method of embodiments 11 or 14, wherein the adverse gastrointestinal event is emesis.
18. Use of the solid dosage form of any one of embodiments 1 to 5 or the pharmaceutical composition of embodiment 6 for treating fibrosis in a subject in need thereof.
19. Use of the solid dosage form of any one of embodiments 1 to 5 or the pharmaceutical composition of embodiment 6 in the manufacture of a medicament for treating fibrosis in a subject in need thereof.
20. The use of embodiment 18 or 19, wherein the fibrosis is a pulmonary fibrosis, in particular idiopathic pulmonary fibrosis; cystic fibrosis; fibrothorax; renal fibrosis; radiation-induced lung injury following treatment for cancer; bridging fibrosis; myocardial fibrosis, in particular interstitial fibrosis or replacement fibrosis; retroperitoneal fibrosis; nephrogenic systemic fibrosis; myelofibrosis; Peyronie's disease; keloid scar; arthrofibrosis; chronic kidney disease; adhesive capsulitis of the shoulder; arthrofibrosis; or systemic sclerosis
21. A solid dosage form of N-1-pyrrolidine-N-5-(3-trifluoromethoxy)phenyl biguanide, or a pharmaceutically acceptable salt thereof, wherein the solid dosage form comprises an enteric coating, for use in treating fibrosis in a subject in need thereof.
22. The solid dosage form of embodiment 21, for use in treating a pulmonary fibrosis, in particular idiopathic pulmonary fibrosis; cystic fibrosis; fibrothorax; renal fibrosis; radiation-induced lung injury following treatment for cancer; bridging fibrosis; myocardial fibrosis, in particular interstitial fibrosis or replacement fibrosis; retroperitoneal fibrosis; nephrogenic systemic fibrosis; myelofibrosis; Peyronie's disease; keloid scar; arthrofibrosis; chronic kidney disease; adhesive capsulitis of the shoulder; arthrofibrosis; or systemic sclerosis in a subject in need thereof.
23. Use of the solid dosage form of any one of embodiments 1 to 5 or the pharmaceutical composition of embodiment 6 for treating cancer in a subject in need thereof.
24. Use of the solid dosage form of any one of embodiments 1 to 5 or the pharmaceutical composition of embodiment 6 in the manufacture of a medicament for treating cancer in a subject in need thereof.
25. The use of embodiment 22 or 23, wherein the cancer is breast cancer, lung cancer, bladder cancer, colon cancer, rectal cancer, uterine cancer, testicular cancer, kidney cancer, leukemia, lymphoma, hepatic cancer, cholangiocarcinoma, melanoma, pancreatic cancer, prostate cancer, thyroid cancer, glioblastoma, or gastric cancer.
26. A solid dosage form of N-1-pyrrolidine-N-5-(3-trifluoromethoxy)phenyl biguanide, or a pharmaceutically acceptable salt thereof, wherein the solid dosage form comprises an enteric coating, for use in treating cancer in a subject in need thereof.
27. The solid dosage form of embodiment 26, for use in treating cancer, in particular breast cancer, lung cancer, bladder cancer, colon cancer, rectal cancer, uterine cancer, testicular cancer, kidney cancer, leukemia, lymphoma, hepatic cancer, cholangiocarcinoma, melanoma, pancreatic cancer, prostate cancer, thyroid cancer, glioblastoma, or gastric cancer.
28. The method of any one of embodiments 10-17, wherein N-1-pyrrolidine-N-5-(3-trifluoromethoxy)phenyl biguanide is administered at a dose of about 100 mg, 200 mg, 400 mg, 800 mg, or 1200 mg per day.
29. The method of embodiment 28, wherein N-1-pyrrolidine-N-5-(3-trifluoromethoxy)phenyl biguanide is administered at a dose of about 800 mg per day.
30. The use or solid dosage form for use of any one of embodiments 18-27, wherein N-1-pyrrolidine-N-5-(3-trifluoromethoxy)phenyl biguanide is to be administered at a dose of about 100 mg, 200 mg, 400 mg, 800 mg, or 1200 mg per day.
31. The use or solid dosage form for use embodiment 30, wherein N-1-pyrrolidine-N-5-(3-trifluoromethoxy)phenyl biguanide is to be administered at a dose of about 800 mg per day.

### Other Embodiments

Various modifications and variations of the described invention will be apparent to those skilled in the art without departing from the scope and spirit of the invention. Although the invention has been described in connection with specific embodiments, it should be understood that the invention as claimed should not be unduly limited to such specific embodiments. Indeed, various modifications of the described modes for carrying out the invention that are obvious to those skilled in the art are intended to be within the scope of the invention.

All references, patents, patent application publications, and patent applications recited herein are hereby incorporated by reference in their entirety.

Other embodiments are in the claims.

What is claimed is:

1. A solid dosage form of N-1-pyrrolidine-N-5-(3-trifluoromethoxy)phenyl biguanide, or a pharmaceutically acceptable salt thereof, wherein the solid dosage form is formulated for delayed release.

2. The solid dosage form of claim 1, wherein the solid dosage form comprises an enteric coating.

3. The solid dosage form of claim 1 or 2, wherein the N-1-pyrrolidine-N-5-(3-trifluoromethoxy)phenyl biguanide, or pharmaceutically acceptable salt thereof, is formulated for release distal to the stomach, to one more regions of the intestine.

4. The solid dosage form of claim 3, in which the onset of N-1-pyrrolidine-N-5-(3-trifluoromethoxy)phenyl biguanide release occurs at or above pH 6 when distal to the stomach.

5. The solid dosage form of any one of claims 1 to 4, wherein the solid dosage form comprises at least one pharmaceutically acceptable excipient.

6. A pharmaceutical composition comprising the solid dosage form of any one of claims 1 to 5.

7. The pharmaceutical composition of claim 6, wherein the pharmaceutical composition is formulated for administration with an anti-emetic agent.

8. The pharmaceutical composition of claim 7, wherein the anti-emetic agent is Ondansetron, Granisetron, Metoclopramide, Domperidone, Promethazine, or Meclizine.

9. The pharmaceutical composition any one of claims 6 to 8, wherein administration of the composition to a subject results in a mean circulating plasma concentration of N-1-pyrrolidine-N-5-(3-trifluoromethoxy)phenyl biguanide that is between 50 and 250 ng/mL for 4-8 hours following administration.

10. A method of treating fibrosis in a subject in need thereof, the method comprising administering an effective amount of the solid dosage form of any one of claims 1 to 5 or the pharmaceutical composition claim 6 or 7.

11. The method of claim 10, wherein the pharmaceutical composition is administered orally.

12. The method of claim 10 or 11, wherein the fibrosis is a pulmonary fibrosis, in particular idiopathic pulmonary fibrosis; cystic fibrosis; fibrothorax; renal fibrosis; radiation-induced lung injury following treatment for cancer; bridging fibrosis; myocardial fibrosis, in particular interstitial fibrosis or replacement fibrosis; retroperitoneal fibrosis; nephrogenic systemic fibrosis; myelofibrosis; Peyronie's disease; keloid scar; arthrofibrosis; chronic kidney disease; adhesive capsulitis of the shoulder; arthrofibrosis; or systemic sclerosis.

13. A method of treating cancer in a subject in need thereof, the method comprising administering an effective amount of the solid dosage form of any one of claims 1 to 5 or the pharmaceutical composition of claim 6 or 7 to the subject.

14. The method of claim 13, wherein the pharmaceutical composition is administered orally.

15. The method of claim 14, wherein the cancer is breast cancer, lung cancer, bladder cancer, colon cancer, rectal cancer, uterine cancer, testicular cancer, kidney cancer, leukemia, lymphoma, hepatic cancer, cholangiocarcinoma, melanoma, pancreatic cancer, prostate cancer, thyroid cancer, glioblastoma, or gastric cancer.

16. The method of any one of claims 10 to 15 wherein administration to a subject does not result in an adverse gastrointestinal event.

17. The method of claim 16, wherein the adverse gastrointestinal event is emesis.

18. The method of any one of claims 10-17, wherein N-1-pyrrolidine-N-5-(3-trifluoromethoxy)phenyl biguanide 1 is administered at a dose of about 100 mg, 200 mg, 400 mg, 800 mg, or 1200 mg per day.

19. The method of claim 18, wherein N-1-pyrrolidine-N-5-(3-trifluoromethoxy)phenyl biguanide is administered at a dose of about 800 mg per day.
